SEQUENCE LISTING (4 sequences)

SEQ ID NO: 1 - DNA, 14117 bp, Bos taurus
ctgccccgacaggcctgacaaccaacaacaagccttcctcaatgccactagagaaatgggaagtgcagaccccttcctgcagcctgctttccacatcctgacttccagattcaggggacatgtccccacactgaggaggctttccttggtagctggaccaggctggttgtggggaggagatacccaaggaataagaacctcccatggccacccccagcccttaggctctagacagggtgagtcaagttgagaagatgaatggcagggctgtgctgggctcagacaaccaaggaacatagactcctgccccagcaaatgcccttggtaaccaggtaggtaggcatgagctaagaggctccaaatctttgcagacatgtggtcaaactggatcagcccagggccagcacagctgtctgcaccctggcaggggacaggcccaccagactccactggtgtggacagcaggaaagcctgacctgcagtagacctgctgcttcagggtgggatcacctgaggtgggcacccccttctggggagcactgtcagccttcataacctcaggatgaaagcccccagtattggtagagcttaggtaggcatcattgcccaatctgcatatgaagagtctgaccctcagggagagaagcagcttgccaagggctgcctttgacttaagccctgctccagttgggcttccctggtggctcagaccctaaagaatctgcctgcaatgtgggaaacctgggttcagtccctgggacgggaagatcccctggagaagggatggcaacccactccagtgttcttgcctgagaatcccacggacagaggagcctggcgggctgcagtccatggagtcgcaaagagtcggacacgactgagcaactaacactttcactttctgccccaataccccacccatctgaacctgaatacctgagtgggtcccactggcaggaagagaggctcctagaggcccagtcctccccaaggctcctcagctttggggcctggattgactgttccaggactctgatgggcggctggggtggatgacgggtagaggctgcctccccagtgactgggacaggcctagccttgtctccacaggtgtccatggacaggactttgcaatccagaggatgggtggtgtggtgcaggctgctgaccactgtgtccagggtcttctctcacgggcccaaggcgcctccaacctggagtcagcccaaggctctttctaaatccccaaacccttccagcccttcattccgccagcctgcagattcctcgtcccaagacagatgttgcttccaccagggggagattcctcattgagctttctttcaacaactcctcacgcacatttgtccccaaaagaccccacctatcttgacgttttccctcgtgcctcttcgctgtgaccctggcagcacctcaatcaggatccagaggtaccagggctgtaggccccgccctccccggaggccccgccctccccggaggccccgccctccccggaggccccgccctccccggaggccccgccctccccggaggccccgccctgtatcaaccttggaccccgtcttcctcaaacaggccccgccccgccttggtacagaggcccttcctgattggtgccttcacagtccgtgccttctcattggcttgaggccctgatctctcaactccagcggtggaacccttggttccctcacgtcccgggtcagatcggttctctttgatgaccctcggcccaccctggtgtcctcactccagctgtttcatgttagccgaaggcaaaggagcctggacgcggacacagggagccgcccccaacacgtaccttcactcgtcagtggctactgtgctcagcctctccaggccaacaggcagcctgagccgtcaatcttctcctctgccaatcagcgcgccagccaggctggccctctagtcagggctcggtactgaaggatggcaagtcccgaaggctcccagggacgcgtgcgcacgggttagggggcttcccaccagctgcctgggagagggatagggagggaaaggcagagctcccgggactcagccctgctgcgcgttcctgagaggactctctcctccttccatcctcccttgggagctatactgagtcctagcgctgagtggcccaactctgcctatgaatagacgaaggtgcttggacactggctaaggggatactcctgatccaccgaggccgggcctgtgaggaggcaagaggggttctccagcctgatgaggtcgctcgagcccttccacacgctactccaagacacgggccaggtagctccagcctgccaggtaaggatgtcaggctggcctcagccgcaaatggtccagtgggagaacatgtcaccagggtcccaggtgcctgttggttgaggtaagagggtcaggagcgagtccggcaggaaggaggcttgatctcaggctgagcctcttggtttatttgctttcagagaggcggtcttcccagctttgcttaccccatgggagtgaacggagtgggttctgtggctaggggtgtttcttgtgtaaaccaggcctaaactcccggtgaaccctcgcatctggagatccaggatactcacactccatgctctttgccaaatgtttgtgaaaccaagtaagatcggccttgcccgcgcacgggcctcactgtgcagttgttttggtgtattggttgcttcattcaacgactggatgactgccgactgtgcaatgaaacagaaacctctgggtccctgcgaatcaacaccccaggatcctaactccctggcaaaactggcccaagtggggaaggcgggaagttctgcaagtctgcagatgaaggcagaagcggggcgggtggagaggcgggctggcttgtctactgtgggggcctgggcaggggagaggtggccaccctgggaataggtgggcatggcacaagtcccggaatgcgaggactgcggcctttctccccctccgttctctgacctggcgcgtgtttgaacagcctaagtggaggaaaagtgggtgcctacggtggtaattagtgggttcacagagcacgaccgtgccgcgggatgtacgttcggtagacgcgttgggtgtcagcctgacgttaacgcactaggcatttcataaataactacaaccccaaattctgcgcctgagctgagaaatgacgaaatcctgtgtttatagagcgggacaaggggcaggcagcggtcagcagaggcttgtttgcagctgcccggaagccccgcgtgttcctcgtctgtccgggattgcatttgccaggagaccacaactcccagggtgcaccgcgcgccagcggactacaaaggtatgcgcgccgcggccctgggccagttagctgctccgggaactacgcttcccaggactccgagaggagccgtccggcacggatttgcacgcgctgattggcggcgcggaccacggcagtggcgtagtagaggcggtggcggcagttggccaagggtccggaggcggggccacaggcctcgggtgctgccagcccggcgggctacgacttggccgcggcggggtgcgaactaaggccatgggcgaccgcggcggcgcgggcggctcccggcgccggaggacggggtcgcggccttcgatccagggcggcagtgggcccgcggcagcggaagaggaggtgcgggatgtgggcgccggaggggacgcgccggtccgggacacagacaaggacggagacgtagacgtgggcagcggccactgggacctgaggtagcggtgcgcgtgacccctaacctttgacccctgatacggggcccctgcgacccaacctggtggcccaggcctgtcggcggcagctcgggctcgagtccgagagtctggcgcctggaccttggtgcacagctgtgcccctcgggcctccacggggaaacttagcgggaggttgggggcggagggtctcctgcccggaacacccaggtacgggggccgaggggagggcagcggctcaacttctagacgccctccctctgccttcctttggtgggttctgaagctttcccagggtgagcccactacgcacagtgtcctctacctggaaggagatacaggggtccttcctgagggctatgaggggtgccttgtgggttgataaagctcccgggggaggagggtggaccggcggagaacagaggcaggggcagtgcgaggggatttctcatccctcgcagaccctccagagaatggtcttcacaaaggtccctcatccgtcacccggcgattgactggcctaggatcctgcttattaccagcacaaatggctgctctagggtcaaagtgggtcctgtaatgggaccctcacccctggttggggtacaggggaggagttggaagtgcgcacacccacaggtgggcgccctgcttagctgaaggactgatgggaaggagttgggggagcaagctgcggctgaaagggaggatctgacccacgtgggcatcagctaagtcctgctggctgcctccaggcgccccctttgccatcctccacgcccctccccccagccctgaccttcatcctggtcaagggctctcaggggctctggttttgggatcagctccagagctagaggttatcaaggaggaagtgggcaacaggtcagtcagcaaggatttgctatcttcactgggtgctgtggggaggggagggacaagggcagttggggtgcaggcactgtccctgcccttggggggcacacagttcacctgagagataagatagccgcagccctgaagagtgagagcaaaggtcaggcacagagttcaggatgacaccaggggagggtggctctgtgaggggcactggcttcctacaggccccaggtggtcctgagggggcggctgcaaaggccaggaggcccacaggcccctctgcccactcctggggaactggatttggggtcactttgtatgaggtgggggcgggtaccagctttgggccaagctgtcaccctggatgggccatcacttgcctgctctgtataggccagatggccagaagctgctcctgtcctgttgatggcccatcctcgaggtctggaccctcgggaagaggagcagttggtggcagggatgggccaccggagaccctcctgacctccaggacacgcagctgtgtgtgcctgtccccaggccacatgccacagggctgggggcctcctggggcagggctgggcattggtctggctactcttggtatcgcctctgcctccctgcctcccagtcatcatcctcccacctctgcctccctgcctgttcctctctttctcctcaggcccttccggacatttcctgctcacctaggtctgggcaggcggggtcaggtgccgggtgtgagctcactccttccggcagcaaggtgtagctatgtgccggaaggaaggccgctgctgttgcctcgcctctgagtgcatcccttccaggtcctccacactcccctgtgccccgacacctggtgcgtccttcagccattggttcatgtgtcctccaggcacagctttctagtccagagcctctaggctgggtgcaggaagtgctgaggaagtggcagccgggaggcgagctggcaccctgtccctccttgttctgtccgtccctggagctggaccgtatggccccgcatgtgtgatccccacttggggctgtgcctctgggcaagttgggaagcttggtgagcctcattttcatgtgcccgcctcccagtactgatgtgcaggttgaatgaggtgccaactgtaatgagttggaatggccctgctggctgggtgggactggggagcaggtgggggccgctggggggcacagaggcacacccagtgcctcagtcagggagagggtgacagagaagctctgggtgaggccccacctccactctggccatggctgctgccctttggtccactgcagtgaactgtgccatggggctggacctctgtggggattggtgggcagtgggctttcttcccgcttggggcctctgacctctgggggcagggcgctgcccgggtgggacagtcggaaggctggtagagggacctgaggggtctgtgtggtggctgggggcaggcctcaggaatttgacagcagggatctggaaaagctttaataacattatttgttgtcaggattgggaaatgctcccctcccccctccccctctttcatcttagagactgctgcacatctggtcagtgtggtcttcttggtggcccccaaggtggcaggggtcacactgttatgaaaccgtcccctgggtatgtggtgcagacatgcacatgcagatggtgattggcaggttgtagcatgaggtggctttgggacggttccagtgacagtgagtgggctggatctggggggttctgggcaggtccatcaagcggatacccccacagactgtcctcttgggatagttgggcctgggagccctgcttgccttgccaaaaggcaggcgcagagtcatgaagaagagggcttgggggctcagagccccactgtgtgtgcagcccagggtggacctggaggaggtgcgtgggcaggctgggccggccggggcctggggtgggggggcctggtgtggcagggaggcagggccagactgtcagcgctgcctggctgaggatgctggcaccctgtcctccccagccgtctgtctcctgggtgcagccatctgagtgctgaccccagccgcccctggaggctggctgttctcctgtgccctattgctggggacatgtgtccacaggagggaaagggaagccccggcctctccccttacaaaactggaggccttgctcaatgccctggatggcctcctggtggcagggtggttggtgggaggtggggctgctgcttagaacccgccagcgggcctgggcctgggctgagctgcacccctccacctctgcctccagctgagggttggcttccatctccaccaggcccagcactgggcacagggctctcagaggcaggctctgaaagtcccctgctggcttctgcagtggactccaggcgccgagcccccagggggctcgcattgcgctcaccctgcgaagccacgtgaaggctgggtcctcccctccggaagggccaaatgcagggcatgggtggtttgaatggtggcccctgggctccccggagggaccagctgctgtgagggccgcccccctccccacttccgtcttgcatcaccagctcctgtggcactccccacgccccgtcccccagtgggagcggcaggcccccggtggctctgcccgcggagggggatgtgtgggcggcggggtggccttgctgccagatgctctgccccgagtgtccgtctccgctctccaggtgtcaccgcctgcaggattccctgttcagttctgacagtggcttcagcaactaccgtggcatcctgaattggtgtgtggtgatgctggtacgtagagtgacaccttggagcaagggtcctgacggccggggggccatgggctcttctccaggggtaggtgtctgtacttgtgtagctgtggtgaatggagctctgtgctggcggtgggggtccctggagcagccgtaccctgggaccctaccgggagcatgctcatgccgtccctgctgaatcccaggagatgcctgcagagggcagcctgggagcctctgagctggggtctgcgccccagggggcactggagtctccccagggggcgagagagagtaggcagggatggtctggtggccctgggtgggggatggctgctccgtgggcccaggccctccctggcagcacaggtgagtggtcttgggggtccacgtagaacttcctcttctgttccaaattgccctcatgggtgcggcatgcctgggtgaacctgggggagcagggtgaggacatgcttctcagcccagcccacagctccaggccacactctgcaggactctggcccctccctcagccctggagggagcaggactggagtcctgtgtccgccttgctctgacctggccgaggccactgctgtggggccccagcaggcctgcccagcagaaggtggagtgcagggaccccaggggcagccttcagggtggggcagggtgaggcccgactgggcccagccccaccgctcagtgctgatgtggcgcgaggccttcgcccctccagctgacgtgtctgcctgccctgggtgtggctccagaggctgcctgtgtaccaggggcccccacgcttctgtttgtggttctgggcagtcccctggggagcggtgggggctgtgtgccagtccagacccagtagtccacgcgtcctggtctctggaggccgtggctggtccaggactgtggcaaggtggtcgtgcagggcaggccctcagcagcctgtctgttctcctgcagcccccagcctcctggccctttggtgcacccacaaagctcccccctcccccaggagctggggccgcctgctgcgtcctctcggcagcctgggcttccaggtggctgggcctcttagcagctccaactcttgcctgtggtgggctctcaggacaggcaactgccagtcggcagacattgcaggaccacgtgtgtcctggtaagctggctggttaggtgtttagctgggggatggtgtggcaggtggcccctgcatctctgagcctgtcacctcctcgggaagccttctgggtgggggactccacccatgtcgcctggagaagcatcacttttccacagagccttctgcaacccccgtggggcctgagcctggggtgggggaggtggtggcccctgctcctgcagaggccagccaggcatctggccccaggccactggcaagagctcgttgtgttgggggatctgtcctttgctgctgctgcaggagcggccgaggcaggcgggggcgtgagtaggggtggagacccaggcccagcttccccagcccctcaggaccggcctgctctttcccaccaccccaccaagtgcgtgggcacaccccgcctgtgaggatgggcccggttggcagggcggagccctgggagggtggcagtgcgccgggcaggcttggacttcactggggcttggggttgtcgctgtggccaggggcgctgacccgcttggtgggacggacggccgctgggcagcaggtttcttctgccacggtggcacaggcacctggggttgtggttggctccaggcgggcgggggctgcgtgcccctgcgcaggcacataggccgtgggtggggagtctcagagcttggcgtgaggtcccacagggctgggcctgcaggatggaggccactgtcctgagctgcaggtgctggcaggagctggggtgggcgttctggggccgtggctgacagcgttatgtccctctctctctatcgcagatcttaagcaacgcacggttatttctagagaacctcatcaagtgagtgggccccggcctgccccagcccctgccacctcacccctcgcctacacagaccctcacccacctgcgtctgcaggtatggcatcctggtggaccccatccaggtggtgtctctgttcctgaaggacccctacagctggccagctctgtgcctggtcattggtgagctgggtgcccaggaggcctcaggccggcggtgggtgggacagggctgatctgggcctgaacctgccctgggttgcttctgtcctcagtggccaatatctttgccgtggctgcgttccaggtggagaagcgcctggccgtggtaagcagtgccctcacgccctcccctgacttgcctcaaggtccttaccagtcgggcttagggcgggccaccagctggtcccactgtgcttcagggttttgggcctttcgtggccttcctgagaggggctgcacctcaggcctggtggctcttcctcagggaggtcctctgaccagggaggggggtccctggctgacgctctgctcccaccccagggagctctgacggagcaggcggggctgctgctgcacggggtcaacctggccaccattctctgcttcccagcggccgtggcctttctcctcgagtctatcactccaggtgggccccacccccgcccccgcccccgcccacgctgtctcggccacgggcagcgcggggggcgtggcctgagcttgcctctcccacagtgggctccgtgctggccctgatggtctacaccatcctcttcctcaagctgttctcctaccgggacgtcaacctctggtgccgagagcgcagggctggggccaaggccaaggctggtgagggctgcctcgggctggggccactgggctgccacttgcctcgggaccggcaggggctcggctcacccccgacccgccccctgccgcttgctcgtagctttggcaggtaaggcggccaacgggggagctgcccagcgcaccgtgagctaccccgacaacctgacctaccgcggtgaggatcctgccgggggctggggggactgcccggcggcctggcctgctagccccgccctcccttccagatctctactacttcctcttcgcccccaccctgtgctacgagctcaacttcccccgctccccccgcatccgaaagcgcttcctgctgcggcgactcctggagatggtgaggcggggcctcgtgggccagggtgggcgggcctgccggcacccggcaccggggctcagctcactgtccgcttgcttccttccccagctgttcctcacccagctccaggtggggctgatccagcaggtacgtgcccgggggggggggggggactctggggccgttggggagctgactctgcgctttttgcagtggatggtcccggccatccagaactccatgaagcccttcaaggtgagcaggcaggcctggcagggtgggttccggggtcagggctgagggagccagctgtgccctgtgcccacaggacatggactactcccgcatcgtggagcgcctcctgaagctggcggtgagtggcctgctgggtggggacgcgtgggggcgggtggggctgttctggcacctggcacccactccccacaggtccccaaccacctcatctggctcatcttcttctactggctcttccactcctgcctgaacgccgtggctgagctcatgcagtttggagaccgcgagttctaccgggactggtggtgggtggccttgccggggcgggggtggtgggggcccccgctggggctggggccggagcccctgcccactctgccccgcccccgcaggaactccgagtccatcacctacttctggcagaactggaacatccctgttcacaagtggtgcatcaggtgggtgtgcgcctgggggcggggggttggggggtgggacggggtcgcgtggcccggcgcccagcccactgccgcctcccccgcagacacttctacaagcccatgctccggcggggcagcagcaagtgggcagccaggacggcagtgtttctggcctccgccttcttccacgaggtcagtgcactgagggcgcgccctgcccctggtgggggtgggggtgggggtgggggctcgctgacgcccctctcccctcagtacctggtgagcatccccctgggaatgttccgcctctgggccttcaccggcatgatggcgcaggtgagcagccctggacccccgctccgccccgccccgcgagcgcagaggctcactcccgtcctgtgtccccagatcccgctggcctggatagtgggccgcttcttccgcggcaactacggcaacgcggccgtgtggctgtcactcatcatcgggcagccggtggccgtcctgatgtacgtccacgactactacgtgctcaaccgtgaggcgccggcagccggcacctgagcgcctccaggctggccccctcgtgggtgttggactgctttgccgcgctgcctgcggctggactagagcctgccccaacctgggtgcagcaggaggaggcctggctggtggaagctgcctcctggcctccaccaggcctctgcctgaagggcttcctcctgccaggggagagcaggcccgacgcagttctggcccctgggaggtgcccatgctctggaaaccctacagatctcgcccaagggtctgaatgtgtcaataaagtgctgtgcacagtgagctccctcagcctccagggcacagggctggcaggagggggcggccctcccacgtggggccatgctgtgggaaggaggccccagcgcctggagaggagctggggctgtggtgaccctccctgcctcacagggctctgtggtcagacgtcttgccctgcaaggtggagactccatgctccaaggccccctgtgcctgaggtctgcacacaagtggattcaacttgggtcaggccagaggctaaggtgtggaagagggttgagaatcaggctgacttgaacggcagcaaagactccaaggcaaggctgcagaggtctcagaggctatgcgcacagtcccctgctggggtgctcacctgggctgggctctgggctgcttggacaaagcaggtggcctggctcagccctcaccgagggcctcccttgggggcagaggttggcctgatgccaggggctccccgtttttccaggccctcagcaggtagttgggtgtggccctcaggataccttggtcccagagcttgccactcaaaaagcttggcagtgaggcaagggcaaccccgggctgttcccccctctactggctctgccgcctgggttggaaaccctgaggctgtgccaggcaggtgtaccctgacagccagccatggcccagtaagatgggtgcccgaggtggtacctgggcagcggacccagctgtgctgcccccgccccaaccagaagccgctctagcccatggtggtcgtctgggcgagacaggctggttggctaggcactgtttggtctacagcaggtgtaggcagcgtctccctgacccctgcctcctaggaagccaccaccctgggccctactcatcagcaaggacagcgagcagggctgagctgggggtgcgtgggctgctacggcccgccacctccatcacatgcacctctgcaccccctgctgcctgactcaggagtgggggggggggtcctgtgcttccttcactccagacccacggtgctgacccagtgcacccacctggtcctctagtgcggacctggccacagggctcctgtgggcccacgctgatcccgccctggtcccttcataaagaactcttgagcacatgcagcccaggggagccaggaggctccagtgtgctgtgtccatctgcctccctccagccccttccgagacactgcgcatcatgcccccctccacccccacccacactggcaggaggaacagacagggagaccacacacagagctcgttgtttataaatctctgcctggctcatcggtctgtttgtccatgtatatatctgtatatctctatggaaggggaaagggggactcgtgtaaaaatccaaaatacaattctatgaacacctgcatcctggtcagtctgagtgtggccgtgaagcccaggtgagctgtggctcacagggctaggccctcggtgctggccgggggccacgccccaccccctctccccccctccgccagccaggggaccaggctcctggacaccaggcctgcccaaggcctgctctcctcctggggcttctacgagacagtggggtccttggctttggggggttctgagcccgtcagcagggagatggtggggtcatccgagtagtcgtctccctcggagaagtaggagccctcccccagctcgaagagcaccggcaggtcgctgctccccacgtccacggagcccgggtccaggagcagcaggggctgggcggtgtagtgcaccaactgcttccctaggggtgcgactgggtcaaggtgccggtggggccggggggcggggtgggggtggggggctcagctcacctgagtctgggctgcttttctctgcctccagaggtctggggggctcctggggagagaggagctcctggatctgctggggcagcaggagggagcacagtgagggctcccgcg

SEQ ID NO: 2 - protein, 489 aa, Bos taurus
MGDRGGAGGSRRRRTGSRPSIQGGSGPAAAEEEVRDVGAGGDAPVRDTDKDGDVDVGSGHWNLRCHRLQDSLFSSDSGFSNYRGILNWCVVMLILSNARLFLENLIKYGILVDPIQVVSLFLKDPYSWPALCLVIVANIFAVAAFQVEKRLAVGALTEQAGLLLHGVNLATILCFPAAVAFLLESITPVGSVLALMVYTILFLKLFSYRDVNLWCRERRAGAKAKAALAGKAANGGAAQRTVSYPDNLTYRDLYYFLFAPTLCYELNFPRSPRIRKRFLLRRLLEMLFLTQLQVGLIQQWMVPAIQNSMKPFKDMDYSRIVERLLKLAVPNHLIWLIFFYWLFHSCLNAVAELMQFGDREFYRDWWNSESITYFWQNWNIPVHKWGIRHFYKPMLRRGSSKWAARTAVFLASAFFHEYLVSIPLRMFRLWAFTGMMAQIPLAWIVGRFFRGNYGNAAVWLSLIIGQPVAVLMYVHDYYVLNREAPAAGT

SEQ ID NO: 3 - DNA, 14117 bp, Bos taurus
ctgccccgacaggcctgacaaccaacaacaagccttcctcaatgccactagagaaatgggaagtgcagaccccttcctgcagcctgctttccacatcctgacttccagattcaggggacatgtccccacactgaggaggctttccttggtagctggaccaggctggttgtggggaggagatacccaaggaataagaacctcccatggccacccccagcccttaggctctagacagggtgagtcaagttgagaagatgaatggcagggctgtgctgggctcagacaaccaaggaacatagactcctgccccagcaaatgcccttggtaaccaggtaggtaggcatgagctaagaggctccaaatctttgcagacatgtggtcaaactggatcagcccagggccagcacagctgtctgcaccctggcaggggacaggcccaccagactccactggtgtggacagcaggaaagcctgacctgcagtagacctgctgcttcagggtgggatcacctgaggtgggcacccccttctggggagcactgtcagccttcataacctcaggatgaaagcccccagtattggtagagcttaggtaggcatcattgcccaatctgcatatgaagagtctgaccctcagggagagaagcagcttgccaagggctgcctttgacttaagccctgctccagttgggcttccctggtggctcagaccctaaagaatctgcctgcaatgtgggaaacctgggttcagtccctgggacgggaagatcccctggagaagggatggcaacccactccagtgttcttgcctgagaatcccacggacagaggagcctggcgggctgcagtccatggagtcgcaaagagtcggacacgactgagcaactaacactttcactttctgccccaataccccacccatctgaacctgaatacctgagtgggtcccactggcaggaagagaggctcctagaggcccagtcctccccaaggctcctcagctttggggcctggattgactgttccaggactctgatgggcggctggggtggatgacgggtagaggctgcctccccagtgactgggacaggcctagccttgtctccacaggtgtccatggacaggactttgcaatccagaggatgggtggtgtggtgcaggctgctgaccactgtgtccagggtcttctctcacgggcccaaggcgcctccaacctggagtcagcccaaggctctttctaaatccccaaacccttccagcccttcattccgccagcctgcagattcctcgtcccaagacagatgttgcttccaccagggggagattcctcattgagctttctttcaacaactcctcacgcacatttgtccccaaaagaccccacctatcttgacgttttccctcgtgcctcttcgctgtgaccctggcagcacctcaatcaggatccagaggtaccagggctgtaggccccgccctccccggaggccccgccctccccggaggccccgccctccccggaggccccgccctccccggaggccccgccctccccggaggccccgccctgtatcaaccttggaccccgtcttcctcaaacaggccccgccccgccttggtacagaggcccttcctgattggtgccttcacagtccgtgccttctcattggcttgaggccctgatctctcaactccagcggtggaacccttggttccctcacgtcccgggtcagatcggttctctttgatgaccctcggcccaccctggtgtcctcactccagctgtttcatgttagccgaaggcaaaggagcctggacgcggacacagggagccgcccccaacacgtaccttcactcgtcagtggctactgtgctcagcctctccaggccaacaggcagcctgagccgtcaatcttctcctctgccaatcagcgcgccagccaggctggccctctagtcagggctcggtactgaaggatggcaagtcccgaaggctcccagggacgcgtgcgcacgggttagggggcttcccaccagctgcctgggagagggatagggagggaaaggcagagctcccgggactcagccctgctgcgcgttcctgagaggactctctcctccttccatcctcccttgggagctatactgagtcctagcgctgagtggcccaactctgcctatgaatagacgaaggtgcttggacactggctaaggggatactcctgatccaccgaggccgggcctgtgaggaggcaagaggggttctccagcctgatgaggtcgctcgagcccttccacacgctactccaagacacgggccaggtagctccagcctgccaggtaaggatgtcaggctggcctcagccgcaaatggtccagtgggagaacatgtcaccagggtcccaggtgcctgttggttgaggtaagagggtcaggagcgagtccggcaggaaggaggcttgatctcaggctgagcctcttggtttatttgctttcagagaggcggtcttcccagctttgcttaccccatgggagtgaacggagtgggttctgtggctaggggtgtttcttgtgtaaaccaggcctaaactcccggtgaaccctcgcatctggagatccaggatactcacactccatgctctttgccaaatgtttgtgaaaccaagtaagatcggccttgcccgcgcacgggcctcactgtgcagttgttttggtgtattggttgcttcattcaacgactggatgactgccgactgtgcaatgaaacagaaacctctgggtccctgcgaatcaacaccccaggatcctaactccctggcaaaactggcccaagtggggaaggcgggaagttctgcaagtctgcagatgaaggcagaagcggggcgggtggagaggcgggctggcttgtctactgtgggggcctgggcaggggagaggtggccaccctgggaataggtgggcatggcacaagtcccggaatgcgaggactgcggcctttctccccctccgttctctgacctggcgcgtgtttgaacagcctaagtggaggaaaagtgggtgcctacggtggtaattagtgggttcacagagcacgaccgtgccgcgggatgtacgttcggtagacgcgttgggtgtcagcctgacgttaacgcactaggcatttcataaataactacaaccccaaattctgcgcctgagctgagaaatgacgaaatcctgtgtttatagagcgggacaaggggcaggcagcggtcagcagaggcttgtttgcagctgcccggaagccccgcgtgttcctcgtctgtccgggattgcatttgccaggagaccacaactcccagggtgcaccgcgcgccagcggactacaaaggtatgcgcgccgcggccctgggccagttagctgctccgggaactacgcttcccaggactccgagaggagccgtccggcacggatttgcacgcgctgattggcggcgcggaccacggcagtggcgtagtagaggcggtggcggcagttggccaagggtccggaggcggggccacaggcctcgggtgctgccagcccggcgggctacgacttggccgcggcggggtgcgaactaaggccatgggcgaccgcggcggcgcgggcggctcccggcgccggaggacggggtcgcggccttcgatccagggcggcagtgggcccgcggcagcggaagaggaggtgcgggatgtgggcgccggaggggacgcgccggtccgggacacagacaaggacggagacgtagacgtgggcagcggccactgggacctgaggtagcggtgcgcgtgacccctaacctttgacccctgatacggggcccctgcgacccaacctggtggcccaggcctgtcggcggcagctcgggctcgagtccgagagtctggcgcctggaccttggtgcacagctgtgcccctcgggcctccacggggaaacttagcgggaggttgggggcggagggtctcctgcccggaacacccaggtacgggggccgaggggagggcagcggctcaacttctagacgccctccctctgccttcctttggtgggttctgaagctttcccagggtgagcccactacgcacagtgtcctctacctggaaggagatacaggggtccttcctgagggctatgaggggtgccttgtgggttgataaagctcccgggggaggagggtggaccggcggagaacagaggcaggggcagtgcgaggggatttctcatccctcgcagaccctccagagaatggtcttcacaaaggtccctcatccgtcacccggcgattgactggcctaggatcctgcttattaccagcacaaatggctgctctagggtcaaagtgggtcctgtaatgggaccctcacccctggttggggtacaggggaggagttggaagtgcgcacacccacaggtgggcgccctgcttagctgaaggactgatgggaaggagttgggggagcaagctgcggctgaaagggaggatctgacccacgtgggcatcagctaagtcctgctggctgcctccaggcgccccctttgccatcctccacgcccctccccccagccctgaccttcatcctggtcaagggctctcaggggctctggttttgggatcagctccagagctagaggttatcaaggaggaagtgggcaacaggtcagtcagcaaggatttgctatcttcactgggtgctgtggggaggggagggacaagggcagttggggtgcaggcactgtccctgcccttggggggcacacagttcacctgagagataagatagccgcagccctgaagagtgagagcaaaggtcaggcacagagttcaggatgacaccaggggagggtggctctgtgaggggcactggcttcctacaggccccaggtggtcctgagggggcggctgcaaaggccaggaggcccacaggcccctctgcccactcctggggaactggatttggggtcactttgtatgaggtgggggcgggtaccagctttgggccaagctgtcaccctggatgggccatcacttgcctgctctgtataggccagatggccagaagctgctcctgtcctgttgatggcccatcctcgaggtctggaccctcgggaagaggagcagttggtggcagggatgggccaccggagaccctcctgacctccaggacacgcagctgtgtgtgcctgtccccaggccacatgccacagggctgggggcctcctggggcagggctgggcattggtctggctactcttggtatcgcctctgcctccctgcctcccagtcatcatcctcccacctctgcctccctgcctgttcctctctttctcctcaggcccttccggacatttcctgctcacctaggtctgggcaggcggggtcaggtgccgggtgtgagctcactccttccggcagcaaggtgtagctatgtgccggaaggaaggccgctgctgttgcctcgcctctgagtgcatcccttccaggtcctccacactcccctgtgccccgacacctggtgcgtccttcagccattggttcatgtgtcctccaggcacagctttctagtccagagcctctaggctgggtgcaggaagtgctgaggaagtggcagccgggaggcgagctggcaccctgtccctccttgttctgtccgtccctggagctggaccgtatggccccgcatgtgtgatccccacttggggctgtgcctctgggcaagttgggaagcttggtgagcctcattttcatgtgcccgcctcccagtactgatgtgcaggttgaatgaggtgccaactgtaatgagttggaatggccctgctggctgggtgggactggggagcaggtgggggccgctggggggcacagaggcacacccagtgcctcagtcagggagagggtgacagagaagctctgggtgaggccccacctccactctggccatggctgctgccctttggtccactgcagtgaactgtgccatggggctggacctctgtggggattggtgggcagtgggctttcttcccgcttggggcctctgacctctgggggcagggcgctgcccgggtgggacagtcggaaggctggtagagggacctgaggggtctgtgtggtggctgggggcaggcctcaggaatttgacagcagggatctggaaaagctttaataacattatttgttgtcaggattgggaaatgctcccctcccccctccccctctttcatcttagagactgctgcacatctggtcagtgtggtcttcttggtggcccccaaggtggcaggggtcacactgttatgaaaccgtcccctgggtatgtggtgcagacatgcacatgcagatggtgattggcaggttgtagcatgaggtggctttgggacggttccagtgacagtgagtgggctggatctggggggttctgggcaggtccatcaagcggatacccccacagactgtcctcttgggatagttgggcctgggagccctgcttgccttgccaaaaggcaggcgcagagtcatgaagaagagggcttgggggctcagagccccactgtgtgtgcagcccagggtggacctggaggaggtgcgtgggcaggctgggccggccggggcctggggtgggggggcctggtgtggcagggaggcagggccagactgtcagcgctgcctggctgaggatgctggcaccctgtcctccccagccgtctgtctcctgggtgcagccatctgagtgctgaccccagccgcccctggaggctggctgttctcctgtgccctattgctggggacatgtgtccacaggagggaaagggaagccccggcctctccccttacaaaactggaggccttgctcaatgccctggatggcctcctggtggcagggtggttggtgggaggtggggctgctgcttagaacccgccagcgggcctgggcctgggctgagctgcacccctccacctctgcctccagctgagggttggcttccatctccaccaggcccagcactgggcacagggctctcagaggcaggctctgaaagtcccctgctggcttctgcagtggactccaggcgccgagcccccagggggctcgcattgcgctcaccctgcgaagccacgtgaaggctgggtcctcccctccggaagggccaaatgcagggcatgggtggtttgaatggtggcccctgggctccccggagggaccagctgctgtgagggccgcccccctccccacttccgtcttgcatcaccagctcctgtggcactccccacgccccgtcccccagtgggagcggcaggcccccggtggctctgcccgcggagggggatgtgtgggcggcggggtggccttgctgccagatgctctgccccgagtgtccgtctccgctctccaggtgtcaccgcctgcaggattccctgttcagttctgacagtggcttcagcaactaccgtggcatcctgaattggtgtgtggtgatgctggtacgtagagtgacaccttggagcaagggtcctgacggccggggggccatgggctcttctccaggggtaggtgtctgtacttgtgtagctgtggtgaatggagctctgtgctggcggtgggggtccctggagcagccgtaccctgggaccctaccgggagcatgctcatgccgtccctgctgaatcccaggagatgcctgcagagggcagcctgggagcctctgagctggggtctgcgccccagggggcactggagtctccccagggggcgagagagagtaggcagggatggtctggtggccctgggtgggggatggctgctccgtgggcccaggccctccctggcagcacaggtgagtggtcttgggggtccacgtagaacttcctcttctgttccaaattgccctcatgggtgcggcatgcctgggtgaacctgggggagcagggtgaggacatgcttctcagcccagcccacagctccaggccacactctgcaggactctggcccctccctcagccctggagggagcaggactggagtcctgtgtccgccttgctctgacctggccgaggccactgctgtggggccccagcaggcctgcccagcagaaggtggagtgcagggaccccaggggcagccttcagggtggggcagggtgaggcccgactgggcccagccccaccgctcagtgctgatgtggcgcgaggccttcgcccctccagctgacgtgtctgcctgccctgggtgtggctccagaggctgcctgtgtaccaggggcccccacgcttctgtttgtggttctgggcagtcccctggggagcggtgggggctgtgtgccagtccagacccagtagtccacgcgtcctggtctctggaggccgtggctggtccaggactgtggcaaggtggtcgtgcagggcaggccctcagcagcctgtctgttctcctgcagcccccagcctcctggccctttggtgcacccacaaagctcccccctcccccaggagctggggccgcctgctgcgtcctctcggcagcctgggcttccaggtggctgggcctcttagcagctccaactcttgcctgtggtgggctctcaggacaggcaactgccagtcggcagacattgcaggaccacgtgtgtcctggtaagctggctggttaggtgtttagctgggggatggtgtggcaggtggcccctgcatctctgagcctgtcacctcctcgggaagccttctgggtgggggactccacccatgtcgcctggagaagcatcacttttccacagagccttctgcaacccccgtggggcctgagcctggggtgggggaggtggtggcccctgctcctgcagaggccagccaggcatctggccccaggccactggcaagagctcgttgtgttgggggatctgtcctttgctgctgctgcaggagcggccgaggcaggcgggggcgtgagtaggggtggagacccaggcccagcttccccagcccctcaggaccggcctgctctttcccaccaccccaccaagtgcgtgggcacaccccgcctgtgaggatgggcccggttggcagggcggagccctgggagggtggcagtgcgccgggcaggcttggacttcactggggcttggggttgtcgctgtggccaggggcgctgacccgcttggtgggacggacggccgctgggcagcaggtttcttctgccacggtggcacaggcacctggggttgtggttggctccaggcgggcgggggctgcgtgcccctgcgcaggcacataggccgtgggtggggagtctcagagcttggcgtgaggtcccacagggctgggcctgcaggatggaggccactgtcctgagctgcaggtgctggcaggagctggggtgggcgttctggggccgtggctgacagcgttatgtccctctctctctatcgcagatcttaagcaacgcacggttatttctagagaacctcatcaagtgagtgggccccggcctgccccagcccctgccacctcacccctcgcctacacagaccctcacccacctgcgtctgcaggtatggcatcctggtggaccccatccaggtggtgtctctgttcctgaaggacccctacagctggccagctctgtgcctggtcattggtgagctgggtgcccaggaggcctcaggccggcggtgggtgggacagggctgatctgggcctgaacctgccctgggttgcttctgtcctcagtggccaatatctttgccgtggctgcgttccaggtggagaagcgcctggccgtggtaagcagtgccctcacgccctcccctgacttgcctcaaggtccttaccagtcgggcttagggcgggccaccagctggtcccactgtgcttcagggttttgggcctttcgtggccttcctgagaggggctgcacctcaggcctggtggctcttcctcagggaggtcctctgaccagggaggggggtccctggctgacgctctgctcccaccccagggagctctgacggagcaggcggggctgctgctgcacggggtcaacctggccaccattctctgcttcccagcggccgtggcctttctcctcgagtctatcactccaggtgggccccacccccgcccccgcccccgcccacgctgtctcggccacgggcagcgcggggggcgtggcctgagcttgcctctcccacagtgggctccgtgctggccctgatggtctacaccatcctcttcctcaagctgttctcctaccgggacgtcaacctctggtgccgagagcgcagggctggggccaaggccaaggctggtgagggctgcctcgggctggggccactgggctgccacttgcctcgggaccggcaggggctcggctcacccccgacccgccccctgccgcttgctcgtagctttggcaggtaagaaggccaacgggggagctgcccagcgcaccgtgagctaccccgacaacctgacctaccgcggtgaggatcctgccgggggctggggggactgcccggcggcctggcctgctagccccgccctcccttccagatctctactacttcctcttcgcccccaccctgtgctacgagctcaacttcccccgctccccccgcatccgaaagcgcttcctgctgcggcgactcctggagatggtgaggcggggcctcgtgggccagggtgggcgggcctgccggcacccggcaccggggctcagctcactgtccgcttgcttccttccccagctgttcctcacccagctccaggtggggctgatccagcaggtacgtgcccgggggggggggggggactctggggccgttggggagctgactctgcgctttttgcagtggatggtcccggccatccagaactccatgaagcccttcaaggtgagcaggcaggcctggcagggtgggttccggggtcagggctgagggagccagctgtgccctgtgcccacaggacatggactactcccgcatcgtggagcgcctcctgaagctggcggtgagtggcctgctgggtggggacgcgtgggggcgggtggggctgttctggcacctggcacccactccccacaggtccccaaccacctcatctggctcatcttcttctactggctcttccactcctgcctgaacgccgtggctgagctcatgcagtttggagaccgcgagttctaccgggactggtggtgggtggccttgccggggcgggggtggtgggggcccccgctggggctggggccggagcccctgcccactctgccccgcccccgcaggaactccgagtccatcacctacttctggcagaactggaacatccctgttcacaagtggtgcatcaggtgggtgtgcgcctgggggcggggggttggggggtgggacggggtcgcgtggcccggcgcccagcccactgccgcctcccccgcagacacttctacaagcccatgctccggcggggcagcagcaagtgggcagccaggacggcagtgtttctggcctccgccttcttccacgaggtcagtgcactgagggcgcgccctgcccctggtgggggtgggggtgggggtgggggctcgctgacgcccctctcccctcagtacctggtgagcatccccctgggaatgttccgcctctgggccttcaccggcatgatggcgcaggtgagcagccctggacccccgctccgccccgccccgcgagcgcagaggctcactcccgtcctgtgtccccagatcccgctggcctggatagtgggccgcttcttccgcggcaactacggcaacgcggccgtgtggctgtcactcatcatcgggcagccggtggccgtcctgatgtacgtccacgactactacgtgctcaaccgtgaggcgccggcagccggcacctgagcgcctccaggctggccccctcgtgggtgttggactgctttgccgcgctgcctgcggctggactagagcctgccccaacctgggtgcagcaggaggaggcctggctggtggaagctgcctcctggcctccaccaggcctctgcctgaagggcttcctcctgccaggggagagcaggcccgacgcagttctggcccctgggaggtgcccatgctctggaaaccctacagatctcgcccaagggtctgaatgtgtcaataaagtgctgtgcacagtgagctccctcagcctccagggcacagggctggcaggagggggcggccctcccacgtggggccatgctgtgggaaggaggccccagcgcctggagaggagctggggctgtggtgaccctccctgcctcacagggctctgtggtcagacgtcttgccctgcaaggtggagactccatgctccaaggccccctgtgcctgaggtctgcacacaagtggattcaacttgggtcaggccagaggctaaggtgtggaagagggttgagaatcaggctgacttgaacggcagcaaagactccaaggcaaggctgcagaggtctcagaggctatgcgcacagtcccctgctggggtgctcacctgggctgggctctgggctgcttggacaaagcaggtggcctggctcagccctcaccgagggcctcccttgggggcagaggttggcctgatgccaggggctccccgtttttccaggccctcagcaggtagttgggtgtggccctcaggataccttggtcccagagcttgccactcaaaaagcttggcagtgaggcaagggcaaccccgggctgttcccccctctactggctctgccgcctgggttggaaaccctgaggctgtgccaggcaggtgtaccctgacagccagccatggcccagtaagatgggtgcccgaggtggtacctgggcagcggacccagctgtgctgcccccgccccaaccagaagccgctctagcccatggtggtcgtctgggcgagacaggctggttggctaggcactgtttggtctacagcaggtgtaggcagcgtctccctgacccctgcctcctaggaagccaccaccctgggccctactcatcagcaaggacagcgagcagggctgagctgggggtgcgtgggctgctacggcccgccacctccatcacatgcacctctgcaccccctgctgcctgactcaggagtgggggggggggtcctgtgcttccttcactccagacccacggtgctgacccagtgcacccacctggtcctctagtgcggacctggccacagggctcctgtgggcccacgctgatcccgccctggtcccttcataaagaactcttgagcacatgcagcccaggggagccaggaggctccagtgtgctgtgtccatctgcctccctccagccccttccgagacactgcgcatcatgcccccctccacccccacccacactggcaggaggaacagacagggagaccacacacagagctcgttgtttataaatctctgcctggctcatcggtctgtttgtccatgtatatatctgtatatctctatggaaggggaaagggggactcgtgtaaaaatccaaaatacaattctatgaacacctgcatcctggtcagtctgagtgtggccgtgaagcccaggtgagctgtggctcacagggctaggccctcggtgctggccgggggccacgccccaccccctctccccccctccgccagccaggggaccaggctcctggacaccaggcctgcccaaggcctgctctcctcctggggcttctacgagacagtggggtccttggctttggggggttctgagcccgtcagcagggagatggtggggtcatccgagtagtcgtctccctcggagaagtaggagccctcccccagctcgaagagcaccggcaggtcgctgctccccacgtccacggagcccgggtccaggagcagcaggggctgggcggtgtagtgcaccaactgcttccctaggggtgcgactgggtcaaggtgccggtggggccggggggcggggtgggggtggggggctcagctcacctgagtctgggctgcttttctctgcctccagaggtctggggggctcctggggagagaggagctcctggatctgctggggcagcaggagggagcacagtgagggctcccgcg

SEQ ID NO: 4 - protein, 489 aa, Bos taurus
MGDRGGAGGSRRRRTGSRPSIQGGSGPAAAEEEVRDVGAGGDAPVRDTDKDGDVDVGSGHWNLRCHRLQDSLFSSDSGFSNYRGILNWCVVMLILSNARLFLENLIKYGILVDPIQVVSLFLKDPYSWPALCLVIVANIFAVAAFQVEKRLAVGALTEQAGLLLHGVNLATILCFPAAVAFLLESITPVGSVLALMVYTILFLKLFSYRDVNLWCRERRAGAKAKAALAGKKANGGAAQRTVSYPDNLTYRDLYYFLFAPTLCYELNFPRSPRIRKRFLLRRLLEMLFLTQLQVGLIQQWMVPAIQNSMKPFKDMDYSRIVERLLKLAVPNHLIWLIFFYWLFHSCLNAVAELMQFGDREFYRDWWNSESITYFWQNWNIPVHKWGIRHFYKPMLRRGSSKWAARTAVFLASAFFHEYLVSIPLRMFRLWAFTGMMAQIPLAWIVGRFFRGNYGNAAVWLSLIIGQPVAVLMYVHDYYVLNREAPAAGT

(19) United States
(12) Patent Application Publication
(10) Pub. No.: US 2004/0234986 A1
(43) Pub. Date: Nov. 25, 2004

(54) Method of testing a mammal for its predisposition for fat content of milk and/ or its predisposition for meat marbling

(76) Inventors: Hans-Rudolf Fries, Freising (DE); Andreas Winter, Gersthofen (DE)

Correspondence Address: Louis M Heidelberger, Reed Smith, 2500 One Liberty Place, 1650 Market Street, Philadelphia, PA, 19103-7301, US

(21) Appl. No.: 10/482,936

(22) PCT Filed: Jul. 5, 2002
(86) PCT No.: PCT/EP02/07520

Related U.S. Application Data
(60) Provisional application No. 60/379,412, filed on May 13, 2002.

(30) Foreign Application Priority Data
Jul. 6, 2001 (EP) 01116412.6

(51) Int. Cl. C12Q 1/68; C07H 21/04; C12N 9/10

(52) U.S. Cl. 435/6; 435/69.1; 435/193; 435/320.1; 435/325; 536/23.2

(57) ABSTRACT

The present invention relates to a newly identified nucleic acid sequence of an allele of the polymorphic bovine DGAT gene. Moreover, the present invention relates to a method of testing a mammal for its predisposition for fat content of milk and/or its predisposition for meat marbling.

The present invention relates to a newly identified nucleic acid sequence of an allele of the polymorphic bovine DGAT gene. Moreover, the present invention relates to a method of testing a mammal for its predisposition for fat content of milk and/or its predisposition for meat marbling.

Several documents are cited throughout the text of this specification. The disclosure content of the documents cited herein (including any manufacture's specifications, instructions, etc.) is herewith incorporated by reference.

Milk fat content is a continuously distributed trait with heritability estimates between 0.45 and 0.50 (Goddard and Wiggans, 1999). There are considerable differences in the average milk fat content between different cattle breeds, ranging from 3.6% in the Holstein to 4.6% in the Jersey breed. The systematic mapping of quantitative trait loci (QTL) underlying the genetic variance of milk production traits resulted in approximate map positions of QTL for milk fat content (Georges et al., 1995; Zhang et al., 1998; Heyen et al., 1999; Velmala et al., 1999). The most consistent results were reported for a QTL on chromosome 14 (Coppieters et al., 1998) (Riquet et al., 1999). The mapping interval of this QTL could be reduced to a few Centimorgans. High-resolution comparative maps of the critical region did not real obvious positional candidate genes (Riquet et al., 1999). DGAT, the gene encoding acyl CoA:diacylglycerol transferase, a microsomal enzyme that catalyses the final step of triglyceride synthesis, became a functional candidate after it had been shown that mice lacking both copies of DGAT show defective lactation. This is most likely the consequence of deficient triglyceride synthesis in the mammary gland (Smith et al., 2000).

Another candidate was reported by Barendse et al. (1999). They described a polymorphism in the 5′ untranslated region of the gene encoding thyroglobulin (TG) which was postulated to be associated with lipid metabolism, particularly the deposition of fat in muscular tissue. Said deposition of fat produces the typical marbling of the meat. The gene was localized on bovine chromosome 14 very close to the DGAT locus (Threadgill et al. 1990). However, the protein encoded by the gene TG is not involved in triglyceride synthesis and thus fat deposition. In summary, the state of the art did so far not provide any genetic link with fat content in milk that can be efficently used in routine testing.

Thus and in of the above, the technical problem underlying the present invention was to provide a method of testing mammals for their predisposition for fat content of milk and/or its predisposition for meat marbling. Said method ought to be easy to use and offer the opportunity to conveniently analyze large nunbers of samples. The solution to this technical problem is achieved by providing the embodiments characterized in the claims.

Accordingly the present invention relates to a nucleic acid molecule encoding a bovine acyl CoA:diacylglycerol transferase (DGAT) contributing to or indicative for low fat content of milk and to low meat marbling (intramuscular fat content); wherein said nucleic acid molecule is selected from the group consisting of:

(a) a nucleic acid molecule having or comprising the nucleic acid sequence of SEQ ID NO: 1;

(b) a nucleic acid molecule comprising the coding sequence of the polypeptide of SEQ ID NO: 2;

(c) a nucleic acid molecule the complementary strand of which hybridizes under stringent conditions to the nucleic acid molecule of (a) or (b), wherein said nucleic acid molecule has at the position corresponding to position 10433 and 10434 of the DGAT gene (SEQ ID NO: 1) a guanine and a cytosine residue; and

(d) a nucleic acid molecule the complementary strand of which hybridizes under stringent conditions to the nucleic acid molecule of (a) or (b), wherein said nucleic acid molecule has at the DGAT gene (SEQ ID NO: 1) position

(i) 3343 a cytosine, 10433 a guanine, 10434 a cytosine, 11030 a guanine, 11048 a cytosine and 11093 a thymine;

(ii) 3343 a cytosine, 10433 a guanine, 10434 a cytosine, 11030 a guanine, 11048 a thymine, and 11093 a thymine; or

(iii) 3343 a guanine, 10433 a guanine, 10434 a cytosine, 11030 a guanine, 11048 a thymine and 11093 a thymine.

Genetic screening (also called genotyping or molecular screening), can be broadly defined as testing to determine if an individual has mutations (alleles or polymorphisms) that either cause a specific phenotype or are “linked” to the mutation causing the phenotype. Linkage refers to the phenomenon that the DNA sequences which are close together in the genome have a tendency to be inherited together. Two or more sequences may be linked because of some selective advantage of co-inheritance. More typically, however, two or more polymorphic sequences are co-inherited because of the relative infrequency with which meiotic recombination events occur within the region between the two polymorphisms. The co-inherited polymorphic alleles are said to be in linkage disequilibrium with one another because, in a given population, they tend to either both occur together or else not occur at all in any particular member of the population. Indeed, where multiple polymorphisms in a given chromosomal region are found to be in linkage disequilibrium with one another, they define a quasi-stable genetic “haplotype.” Furthermore, where a phenotype-causing mutation is found within or in linkage with this haplotype, one or more polymorphic alleles of the haplotype can be used as a diagnostic or prognostic indicator of the likelihood of developing a specific phenotype. Identification of a haplotype which spans or is linked to a phenotype-causing mutational change, serves as a predictive measure of an individual's likelihood of having inherited that phenotype-causing mutation. Importantly, such prognostic or diagnostic procedures can be utilized without necessitating the identification and isolation of the actual phenotype-causing molecule. This is significant because the precise determination of the molecular basis of the establishment of a specific phenotype can be difficult and laborious, especially in the case of multifactorial phenotype.

Mapping studies on human chromosome 8 placed DGAT indirectly within the mapping interval of the QTL on bovine chromosome 14, the homologous counterpart of human chromosome 8. Sequencing of DGAT from pooled DNA revealed massive frequency shifts at several variable positions between groups of animals with high and low milk fat percentage, respectively. The procedure of said sequencing is described in example 6. It was searched for variation in 10528 basepairs, i.e., the entire coding region of DGAT, the major part of the introns and the 5′ and 3′ regions. 20 variable positions were identified, mostly single nucleotide polymorphisms (summerized in table 9). By said method several nucleotide polymorphisms were detected which were unexpected vis-á-vis the prior art data for the sequences known from the region the DGAT in mice, human or plants. Among the variants is a double substitution causing the non-conservative substitution of alanine by lysine. Furthermore, said variants comprised several single nucleotide substitutions. An example for a sequence containing said newly identified polymorphisms is SEQ ID NO: 1.

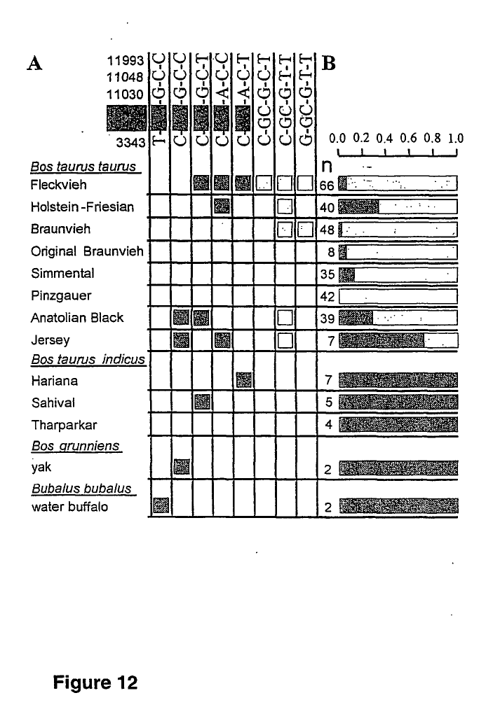

Direct sequencing in animals belonging to different breeds of Bos taurus taurus and Bos taurus indicus as well as in animals of Bos grunniens (yak) and Bubalus bubalus (water buffalo) at position 3343, 10433, 10434, 11030, 11048 and 11093 allowed to derive at least 8 haplotypes (see FIG. 12). The haplotypes observed encoded a DGAT1 protein with either a lysine or an alanine in position 232 of the DGAT1 polypeptide sequence. In addition, specific nucleotides at positions 3343, 10433, 10434, 11030, 11048 and 11093 were demonstrated to be indicative of a specific haplotype. As shown in FIG. 12A, haplotypes encoding a protein with a lysine in position 232 may contain in the above mentioned positions either TMGCC, CAAGCC, CMGCT, CAMCC or CAAACT while alanine encoding haplotypes are characterized by CGCGCT (i.e. at position: 3343 cytosine, 10433 a guanine, 10434 a cytosine, 11030 a guanine, 11048 a cytosine and 11093 a thymine), CGCGTT (i.e. at position: 3343 a cytosine, 10433 a guanine, 10434 a cytosine, 11030 a guanine, 11048 a thymine, and 11093 a thymine) or GGCGTT (i.e. at position: 3343 a guanine, 10433 a guanine, 10434 a cytosine, 11030 a guanine, 11048 a thymine and 11093 a thymine) in the above mentioned positions. It is of note that the invention also comprises sequences wherein one or two nucleotides in the above-indicated positions are exchanged by different nucleotides. In addition, the invention comprises haplotypes arising from recombination events and including the above recited gene.

Furthermore, an RFLP analysis revealed frequency estimates for lysine and alanine encoding alleles in several cattle breeds of Bovinae subfamilies (see FIG. 12b). Distinct frequency differences for the allelic distribution in various breeds indicated a correlation between milk fat content and the genetic variation.

The term “hybridizes under stringent conditions”, as used in the description of the present invention, is well known to the skilled artisian and corresponds to conditions of high stringency. Appropriate stringent hybridization conditions for each sequence may be established by a person skilled in the art on well-known parameters such as temperature, composition of the nucleic acid molecules, salt conditions etc.; see, for example, Sambrook et al., “Molecular Cloning, A Laboratory Manual”; CSH Press, Cold Spring Harbor, 1989 or Higgins and Hames (eds.), “Nucleic acid hybridization, a practical approach”, IRL Press, Oxford 1985, see in particular the chapter “Hybridization Strategy” by Britten & Davidson, 3 to 15. Stringent hybridization conditions are, for example, conditions comprising overnight incubation at 42° C. in a solution comprising: 50% formamide, 5×SSC (750 mM NaCl, 75 mM trisodium citrate), 50 mM sodium phosphate (pH 7.6), 5× Denhardt's solution, 10% dextran sulfate, and 20 micrograms/ml denatured, sheared salmon sperm DNA, followed by washing the filters in 0.1×SSC at about 65°. Other stringent hybridization conditions are for example 0.2×SSC (0.03 M NaCl, 0.003M Natriumcitrat, pH 7) bei 65° C. Preferred in accordance with the present invention are nucleic acids which are capable of hybridizing to the nucleic acid molecule of the invention or parts thereof wherein said nucleic acid molecule has at the position corresponding to position 10433 and 10434 of the DGAT gene (SEQ ID NO: 1) a guanine and a cytosine residue. More preferred in accordance with the present invention are nucleic acids which are capable of hybridizing to the complementary strand of any of the nucleic acid molecules of the invention or parts thereof, wherein said nucleic acid molecule contains at position 3343, 10433, 10434, 11030, 11048 and 11093 of the DGAT gene (SEQ ID NO: 1) nucleotides which are either CGCGCT, CGCGTT or GGCGTT. Furthermore, the nucleic acid molecules of the invention may contain any alanine codon at the position encoding amino acid 232 of DGAT.

The term “corresponding” as used herein means that a position is not only determined by the number of the preceding nucleotides and amino acids, respectively. The position of a given nucleotide or amino acid in accordance with the present invention which may be deleted, substituted or comprise one or more additional nucleotide(s) may vary due to deletions or additional nucleotides or amino acids elsewhere in the gene or the polypeptide. Thus, under a “corresponding position” in accordance with the present invention it is to be understood that nucleotides or amino acids may differ in the indicated number but may still have similar neighboring nucleotides or amino acids. Said nucleotides or amino acids may for instance together with their neighbors form sequences which may be involved in the regulation of gene expression, stability of the corresponding RNA or RNA editing, as well as encode functional domains or motifs of the protein of the invention. In the context of the invention functional domains or motifs of the invention are defined as portions having the enzymatic activity of DGAT and/or portions which are capable to be recognized as an antigen and therefore represent an epitope for an antibody or small molecule.

Therefore, the invention comprises allelic variants of the DGAT gene as well as recombinantly or otherwise altered DGAT sequences. In conformance with the present invention, the recited nucleic acid “encodes” the DGAT enzyme. Whereas by definition the claimed nucleic acid molecule comprises the coding region, it may also comprise non-coding regions such as regulatory reigns or introns.

Apart from being the subject of investigation, the nucleic acid molecule of the invention may be useful as probes in Northern or Southern Blot analysis of RNA or DNA preparations, respectively, or can be used as oligonucleotide primers in PCR analysis dependent on their respective size. Also comprised by the invention are hybridizing nucleic acids which are useful for analyzing DNA-Protein interactions via, e.g., electrophoretic mobility shift analysis (EMSA). Preferably, said hybridizing nucleic acids comprise at least 10, more preferably at least 15 nucleotides in length while a hybridizing polynucleotide of the present invention to be used as a probe preferably comprises at least 100, more preferably at least 200, or most preferably at least 500 nucleotides in length.

The nucleic acid molecule of the invention is expected to occur in any breed of the bovine species. In a preferred embodiment of the invention the bovine nucleic acid molecule is a nucleic acid molecule of a bovine animal selected from the group consisting of Ayrshire, Bazadaise, Beefalo, Blaarkop, Braunvieh Fleischnutzung, Grauvieh, Lakenfelder, Limpurger Fleischnutzung, Maine Anjou, Marchigiana, Montbeliard, Murnau-Werdenfelser, Normanne, Romagnola, Rotbunt Fleischnutzung, Telemark, Tuxer, Vogesen-Rind, Wasserbüffel, Witrug, Yak, Auerochse, Bison/Wisent, Hinterwälder Fleischnutzung, Vorderwälder Fleischnutzung, Angler, Doppelnutzung Rotbunt, Holstein-Rbt., Holstein-Sbt., Holstein-Friesian, Deutsches Shorthorn, Rotvieh alter Angler, Aberdeen Angus, Aubrac, Blonde d'Aqultaine, Brahman, Brangus, Charolais, Chlanina, Deutsche Angus, Fjall-Rind, Fleckvieh Fleischnutzung Ost, Gelbvieh Fleischnutzung, Hereford, Jersey, Limousin, Lincoln Red, Piemonteser, Salers, South Devon, Weifblaue Belgier, Beited Galloway, Dexter, Galloway, Highland, Longhorn, Luing, Ungarisches Steppenrind, Welsh-Black, White Galloway, White Park, Zwerg-Zebus, Rotvieh Zuchtrichtung, Uckermärker, Deutsche Schwarzbunte alter, Braunvieh, Fleckvieh, Gelbvieh, Pinzgauer Fleischnutzung, Ansbach-Triesdorfer, Braunvieh alter Zuchtrichtung, Limpurger, Murnau-Werdenfelser, Pinzgauer, Pustertaler Schecken, Hinterwäldler, Vorderwäldler and Glanrind.

In a more preferred embodiment of the invention the bovine nucleic acid molecule is a nucleic acid molecule of a female bovine animal.

The nucleic acid molecule can be taken from any nucleic acid containing tissue. Preferably said nucleic acid molecule is present in a sample taken from, for example, from muscle, blood, skin, milk, urine and other samples taken from a bovine animal.

Preferably said nucleic acid molecule is mRNA, genomic DNA (gDNA) or cDNA which is derived from said mRNA by reverse transcription of said mRNA. The method or reverse transcription of mRNA into cDNA is well established and known by a person skilled in the art.

More preferably said gDNA is a gene.

In an preferred embodiment of the invention the nucleic acid molecule is a fragment of the herein above described nucleic acid molecule having at least 14 nucleotides wherein said fragment comprises nucleotide position 10433 and 10434 of SEQ ID NO: 1.

Said nucleic acid molecule may, for example, be used as hybridization probe. For hybridization probes, it may be, e.g., desirable to use nucleic acid analogs, in order to improve the stability and binding affinity. The term “nucleic acid” shall be understood to encompass such analogs. A number of modifications have been described that alter the chemistry of the phosphodiester backbone, sugars or heterocyclic bases. Among useful changes in the backbone chemistry are phosphorothioates; phosphorodithioates, where both of the non-bridging oxygens are substituted with sulfur; phosphoroamidites; alkyl phosphotriesters and boranophosphates. Achiral phosphate derivatives include 3′-O′-5′-S-phosphorothioate, 3′-S-5′-O-phosphorothioate, 3′-CH2-5′-O-phosphonate and 3′-NH-5′-O-phosphoroamidate. Peptide nucleic acids replace the entire phosphodiester backbone with a peptide linkage. Sugar modifications are also used to enhance stability and affinity. The a-anomer of deoxyribose may be used, where the base is inverted with respect to the natural b-anomer. The 2′-OH of the ribose sugar may be altered to form 2′-O-methyl or 2′-O-allyl sugars, which provides resistance to degradation without comprising affinity. Modification of the heterocyclic bases must maintain proper base pairing. Some useful substitutions include deoxyuridine for deoxythymidine; 5-methyl-2′-deoxycytidine and 5-bromo-2′-deoxycytidine for deoxycytidine. 5-propynyl-2′-deoxyuridine and 5-propynyl-2′-deoxycytidine have been shown to increase affinity and biological activity when substituted for deoxythymidine and deoxycytidine, respectively.

The hybridization probe or the primer(s) used for amplification may also contain a detectable label. Suitable labels include fluorochromes, e.g. fluorescein isothiocyanate (FITC), rhodamine, Texas Red, phycoerythrin, allophycocyanin, 6-carboxyfluorescein (6-FAM), 2′,7′-dimethoxy-4′,5′-dichloro-6-carboxyfluorescein (JOE), 6-carboxy-X-rhodamine(ROX), 6-carboxy-2′,4′,7′,4,7-hexachlorofluorescein (HEX), 5-carboxyfluorescein (5-FAM) or N,N,N′,N′-tetramethyl-6-carboxyrhodamine (TAMRA), radioactive labels, e.g. ³²P, ³⁵S, ³H; etc. The label may also be a two stage system, where the DNA is conjugated to biotin, haptens, etc. having a high affinity binding partner, e.g. avidin, specific antibodies, etc., where the binding partner is conjugated to a detectable label. In the case of amplification the label may be conjugated to one or both of the primers. The pool of nucleotides used in the amplification may also be labeled, so as to incorporate the label into the amplification product. Alternatively, the double strand formed after hybridization can be detected by anti-double strand DNA specific antibodies or aptamers etc.

More preferably said nucleic acid molecule is complementary to the above described nucleic acid. Said complementary nucleic acid molecule is suitable to hybridize specifically with a polynucleotide as described above. Specific hybridization occurs preferably under stringent conditions and implies no or very little cross-hybridization with nucleotide sequences encoding no or substantially different proteins. Such nucleic acid molecules may be used as probes and/or for the control of gene expression. Nucleic acid probe technology is well known to those skilled in the art who will readily appreciate that such probes may vary in length. Preferred are nucleic acid probes of 17 to 35 nucleotides in length. Of course, it may also be appropriate to use nucleic acids of up to 100 and more nucleotides in length. The nucleic acid probes of the invention are useful for various applications. On the one hand, they may be used as PCR primers for amplification of nucleic acid molecules according to the invention. Another application is the use as a hybridization probe to identify polynucleotides hybridizing to the nucleic acid molecule of the invention by homology screening of genomic DNA libraries (see example 3). Nucleic acid molecules according to this preferred embodiment of the invention which are complementary to a polynucleotide as described above may also be used for repression of expression of a gene comprising such a polynucleotide, for example due to an antisense or triple helix effect or for the construction of appropriate ribozymes (see, e.g., EP-A1 0 291 533, EP-A1 0 321 201, EP-A2 0 360 257) which specifically cleave the (pre)-mRNA of a gene comprising a polynucleotide of the invention. Selection of appropriate target sites and corresponding ribozymes can be done as described for example in Steinecke, Ribozymes, Methods in Cell Biology 50, Galbraith et al. eds Academic Press, Inc. (1995), 449-460. Standard methods relating to antisense technology have also been described (Melani, Cancer Res. 51 (1991), 2897-2901). Furthermore, the person skilled in the art is well aware that it is also possible to label such a nucleic acid probe with an appropriate marker for specific applications, such as for the detection of the presence of a polynucleotide of the invention in a sample derived from an organism.

The above described nucleic acid molecules may either be DNA or RNA or a hybrid thereof. Furthermore, said nucleic acid molecule may contain, for example, thioester bonds and/or nucleotide analogues, commonly used in oligonucleotide anti-sense approaches. Said modifications may be useful for the stabilization of the nucleic acid molecule against endo- and/or exonucleases in the cell. Said nucleic acid molecules may be transcribed by an appropriate vector containing a chimeric gene which allows for the transcription of said nucleic acid molecule in the cell. Such nucleic acid molecules may further contain ribozyme sequences as described above.

Furthermore, the present invention provides a vector comprising the herein above described nucleic acid molecule. Said expression vectors may particularly be plasmids, cosmids, viruses or bacteriophages used conventionally in genetic engineering plasmids, cosmids, viruses and bacteriophages used conventionally in genetic engineering that comprise the aforementioned nucleic acid. Preferably, said vector is a gene transfer or targeting vector. Expression vectors derived from viruses such as retroviruses, vaccinia virus, adeno-associated virus, herpes viruses, or bovine papilloma virus, may be used for delivery of the nucleic acid into targeted cell population. Methods which are well known to those skilled in the art can be used to construct recombinant viral vectors; see, for example, the techniques described in Sambrook et al., Molecular Cloning A Laboratory Manual, Cold Spring Harbor Laboratory (1989) N.Y. and Ausubel et al., Current Protocols in Molecular Biology, Green Publishing Associates and Wiley Interscience, New York (1989). Alternatively, the nucleic acids and vectors can be reconstituted into liposomes for delivery to target cells. The vectors containing the nucleic acid can be transferred into the host cell by well-known methods, which vary depending on the type of cellular host. For example, calcium phosphate or DEAE-Dextran mediated transfection or electroporation may be used for eukaryotic cellular hosts; see Sambrook, supra. Such vectors may comprise further genes such as marker genes which allow for the selection of said vector in a suitable host cell and under suitable conditions.

Preferably, said vector comprises regulatory elements for expression of said nucleic acid molecule. Consequently, the nucleic acid of the invention may be operatively linked to expression control sequences allowing expression in eukaryotic cells. Expression of said nucleic acid molecule comprises transcription of the sequence nucleic acid molecule into a translatable mRNA. Regulatory elements ensuring expression in eukaryotic cells, preferably mammalian cells, are well known to those skilled in the art. They usually comprise regulatory sequences ensuring initiation of transcription and, optionally, a poly-A signal ensuring termination of transcription and stabilization of the transcript, and/or an intron further enhancing expression of said nucleic acid. Additional regulatory elements may include transcriptional as well as translational enhancers, and/or naturally-associated or heterologous promoter regions. Possible regulatory elements permitting expression in eukaryotic host cells are the AOX1 or GAL1 promoter in yeast or the CMV-, SV40-, RSV-promoter (Rous sarcoma virus), CMV-enhancer, SV40-enhancer or a globin intron in mammalian and other animal cells. Beside elements which are responsible for the initiation of transcription such regulatory elements may also comprise transcription termination signals, such as the SV40-poly-A site or the tk-poly-A site, downstream of the nucleic acid molecule. Furthermore, depending on the expression system used leader sequences capable of directing the polypeptide to a cellular compartment or secreting it into the medium may be added to the coding sequence of the aforementioned nucleic acid and are well known in the art. The leader sequence(s) is (are) assembled in appropriate phase with translation, initiation and termination sequences, and preferably, a leader sequence capable of directing secretion of translated protein, or a portion thereof, into the periplasmic space or extracellular medium. Optionally, the heterologous sequence can encode a fusion protein including an C- or N-terminal identification peptide imparting desired characteristics, e.g., stabilization or simplified purification of expressed recombinant product. In this context, suitable expression vectors are known in the art such as Okayama-Berg cDNA expression vector pcDVI (Pharmacia), pCDM8, pRc/CMV, pcDNA1, pcDNA3, the Echo™ Cloning System (Invitrogen), pSPORT1 (GIBCO BRL) or pRevTet-On/pRevTet-Off or pCl (Promega).

Another preferred embodiment of the invention relates to primer or primer pair, wherein the primer or primer pair hybridize under stringent conditions to the nucleic acid molecule of the invention comprising nucleotide position 10433 and 10434 of SEQ ID NO: 1 or the complement strand thereof. The exact composition of the primer sequences is not critical as long as they allow detection of the desired sequence(s). Preferably, the primers are chosen in such a way that they hybridize under stringent conditions to the desired sequence(s). It is preferable to choose a primer or a pair of primers that will generate an amplification product of at least 50 nt, preferably of at least about 100 nt and most preferably of at least 200 nt. Algorithms for the selection of primer sequences are generally known and are available in commercial software packages (see example 1). Amplification primers hybridize to complementary strands of DNA and will prime towards each other.

Furthermore, the present invention relates to a host cell which contains the herewith above described expression vector.

Preferably, said host cell is a eukaryotic, most preferably a mammalian cell if therapeutic uses of the protein are envisaged. Of course, yeast and less preferred prokaryotic, e.g., bacterial cells may serve as well, in particular if the produced protein is used as a diagnostic means.

The polynucleotide or vector of the invention which is present in the host cell may either be integrated into the genome of the host cell or it may be maintained extrachromosomally.

The term “prokaryotic” is meant to include all bacteria which can be transformed or transfected with a DNA or RNA molecules for the expression of a protein of the invention. Prokaryotic hosts may include gram negative as well as gram positive bacteria such as, for example, E. coli, S. typhimurium, Serratia marcescens and Bacillus subtilis. The term “eukaryotic” is meant to include yeast, higher plant, insect and preferably mammalian cells. Depending upon the host employed in a recombinant production procedure, the protein encoded by the polynucleotide of the present invention may be glycosylated or may be non-glycosylated. A nucleic acid molecule of the invention can be used to transform or transfect the host using any of the techniques commonly known to those of ordinary skill in the art. Furthermore, methods for preparing fused, operably linked genes and expressing them in, e.g., mammalian cells and bacteria are well-known in the art (Sambrook, Molecular Cloning: A Laboratory Manual, Cold Spring Harbor Laboratory, Cold Spring Harbor, N.Y., 1989). The genetic constructs and methods described therein can be utilized for expression of the protein of SEQ ID NO: 2 in eukaryotic or prokaryotic hosts. In general, expression vectors containing promoter sequences which facilitate the efficient transcription of the inserted polynucleotide are used in connection with the host. The expression vector typically contains an origin of replication, a promoter, and a terminator, as well as specific genes which are capable of providing phenotypic selection of the transformed cells.

In an alternative embodiment the present invention relates to a method for production of a functional bovine DGAT or a functional fragment thereof comprising:

(a) culturing said host cell containing the expression vector which comprises the herein above mentioned nucleic acid molecule under conditions allowing the expression of the encoded polypeptide; and

(b) collecting the polypeptide from the culture.

As aforementioned, a functional fragment is defined in the context of the present invention as a fragment having the enzymatic activity of DGAT and/or fragment which is capable to be recognized as an antigen and therefore represent an epitope for an antibody and/or small molecule suitable for specific binding and detection of an epitope.

The transformed hosts can be grown in fermentors and cultured according to techniques known in the art to achieve optimal cell growth. The protein of the invention can then be isolated from the growth medium, cellular lysates, or cellular membrane fractions. Once expressed, the protein of the present invention can be purified according to standard procedures of the art, including ammonium sulfate precipitation, affinity columns, column chromatography, gel electrophoresis and the like; see, Scopes, “Protein Purification”, Springer-Verlag, N.Y. (1982). Substantially pure proteins of at least about 90 to 95% homogeneity are preferred, and 98 to 99% or more homogeneity are most preferred, for pharmaceutical uses. Once purified, partially or to homogeneity as desired, the proteins may then be used therapeutically (including extracorporeally) or in developing and performing assay procedures.

Hence, in a still further embodiment, the present invention relates to functional bovine DGAT polypeptide as depicted in SEQ ID NO: 2 or a functional fragment thereof encoded by a nucleic acid molecule (SEQ ID NO: 1) or produced by a method of as described above. It will be apparent to those skilled in the art that the protein of the invention can be further coupled to other moieties for, e.g., drug targeting and imaging applications. Such coupling may be conducted chemically after expression of the protein to site of attachment or the coupling product may be engineered into the protein of the invention at the DNA level. The DNAs are then expressed in a suitable host system, and the expressed proteins are collected and renatured, if necessary.

Furthermore, the provision of the protein of the present invention enables the production of DGAT specific antibody which binds to an epitope of the polypeptide or fragment of SEQ ID NO: 2 the epitope comprising a alanine at position 232 but not to a polypeptide or a fragment of SEQ ID NO: 4 having a lysine at position 232. In an alternative embodiment the invention relates to the production of DGAT specific antibody which binds to an epitope of the polypeptide or fragment of SEQ ID NO: 4 the epitope comprising a lysine at position 232 but not to a polypeptide or a fragment of SEQ ID NO: 2 having a alanine at position 232.

In this respect, hybridoma technology enables production of cell lines secreting antibody to essentially any desired substance that produces an immune response. RNA encoding the light and heavy chains of the immunoglobulin can then be obtained from the cytoplasm of the hybridoma. The 5′ end portion of the mRNA can be used to prepare cDNA to be inserted into an expression vector. The DNA encoding the antibody or its immunoglobulin chains can subsequently be expressed in cells, preferably mammalian cells.

Depending on the host cell, renaturation techniques may be required to attain proper conformation of the antibody. If necessary, point substitutions seeking to optimize binding may be made in the DNA using conventional cassette mutagenesis or other protein engineering methodology such as is disclosed herein.

Said antibodies, which are monoclonal antibodies, polyclonal antibodies, single chain antibodies, or fragment thereof that specifically binds said peptide or polypeptide also including bispecific antibody, synthetic antibody, antibody fragment, such as Fab, a F(ab₂)′, Fv or scFv fragments etc., or a chemically modified derivative of any of these (all comprised by the term “antibody”). Monoclonal antibodies can be prepared, for example, by the techniques as originally described in Köhler and Milstein, Nature 256 (1975), 495, and Galfré, Meth. Enzymol. 73 (1981), 3, which comprise the fusion of mouse myeloma cells to spleen cells derived from immunized mammals with modifications developed by the art. Furthermore, antibodies or fragments thereof to the aforementioned peptides can be obtained by using methods which are described, e.g., in Harlow and Lane “Antibodies, A Laboratory Manual”, CSH Press, Cold Spring Harbor, 1988. When derivatives of said antibodies are obtained by the phage display technique, surface plasmon resonance as employed in the BIAcore system can be used to increase the efficiency of phage antibodies which bind to an epitope of the peptide or polypeptide of the invention (Schier, Human Antibodies Hybridomas 7 (1996), 97-105; Malmborg, J. Immunol. Methods 183 (1995), 7-13). The production of chimeric antibodies is described, for example, in WO89/09622. A further source of antibodies to be utilized in accordance with the present invention are so-called xenogenic antibodies. The general principle for the production of xenogenic antibodies such as human antibodies in mice is described in, e.g., WO 91/10741, WO 94/02602, WO 96/34096 and WO 96/33735. Antibodies to be employed in accordance with the invention or their corresponding immunoglobulin chain(s) can be further modified using conventional techniques known in the art, for example, by using amino acid deletion(s), insertion(s), substitution(s), addition(s), and/or recombination(s) and/or any other modification(s) known in the art either alone or in combination. Methods for introducing such modifications in the DNA sequence underlying the amino acid sequence of an immunoglobulin chain are well known to the person skilled in the art; see, e.g., Sambrook, Molecular Cloning A Laboratory Manual, Cold Spring Harbor Laboratory (1989) New York.

Moreover, the present invention relates to a transgenic, non-human animal comprising at least the herein above disclosed nucleic acid molecules. Preferably said transgenic, non-human animal belongs to cattle.

In an other embodiment the present invention relates to a method of testing a mammal for its predisposition for fat content of milk and/or its predisposition for meat marbling comprising analyzing the nucleic acid of a sample comprising the gene encoding DGAT, a corresponding mRNA for nucleotide polymorphisms which are connected with said predisposition or any nucleic acid molecule of the invention. The term “its predisposition for fat content of milk and/or its predisposition for meat marbling” describes the capability of a mammal to produce milk with high fat, respectively low fat content and/or its capability to produce meat with high intramuscular fat content, respectively low intramuscular fat content.

Preferably the nucleic acid of said method is DNA.

More preferably the nucleic acid of said method is gDNA (genomic DNA).

Also more preferred the nucleic acid is cDNA which is derived from said mRNA by reverse transcription of said mRNA.

In accordance with the invention the nucleotide polymorphisms which are contributing to or indicative for low fat content of milk and to low meat marbling are in one preferred embodiment located in the coding region of the DGAT gene.

More preferably the nucleotide polymorphisms in the coding region of the gene encoding DGAT result in substitution, deletion and/or addition of at least one amino acid in the amino acid sequence of the polypeptide which is encoded by said gene.

Further more preferably said nucleic acid molecule has at the position corresponding to position 10433 and 10434 of the DGAT gene (SEQ ID NO: 1) a guanine and a cytosine residue which corresponds to i.e. correlates with a predisposition for low fat content of milk and low meat marbling.

More preferably the nucleic acid molecule has at the positions corresponding to position 3343, 10433, 10434, 11030, 11048 and 11093 of the DGAT gene (SEQ ID NO:1) the nucleotides CGCGCT (i.e. at position 3343 a cytosine, 10433 a guanine, 10434 a cytosine, 11030 a guanine, 11048 a cytosine and 11093 a thymine), CGCGTT (i.e. at position 3343 a cytosine, 10433 a guanine, 10434 a cytosine, 11030 a guanine, 11048 a thymine, and 11093 a thymine) or GGCGTT (i.e. at position 3343 a guanine, 10433 a guanine, 10434 a cytosine, 11030 a guanine, 11048 a thymine and 11093 a thymine) which corresponds to i.e. correlates with a predisposition for low fat content of milk and low meat marbling.

Alternatively said nucleic acid molecule has at the position corresponding to position 10433 and 10434 of the DGAT gene (SEQ ID NO: 3) two adenine residue which corresponds to i.e. correlates with a predisposition for high fat content of milk and high meat marbling.

More preferably said nucleic acid molecule has at the positions corresponding to positions 3343, 10433, 10434, 11030, 11048 and 11093 of the DGAT gene the nucleotides TMGCC (i.e. at position 3343 a thymine, 10433 an adenosine, 10434 an adenosine, 11030 a guanine, 11048 a cytosine and 11 093 a cytosine), CAAGCC (i.e. at position 3343 a cytosine, 10433 an adenosine, 10434 an adenosine, 11030 a guanine, 11048 a cytosine, and 11093 a cytosine), CAAGCT (i.e. at position 3343 a cytosine, 10433 an adenosine, 10434 an adenosine, 11030 a guanine, 11048 a cytosine and 11093 a thymine), CAAACC (i.e. at position 3343 a cytosine, 10433 an adenosine, 10434 an adenosine, 11030 an adenosine, 11048 a cytosine and 11093 a cytosine) or CAAACT (i.e. at position 3343 a cytosine, 10433 an adenosine, 10434 an adenosine, 11 030 an adenosine, 11048 a cytosine and 11093 a thymine) which corresponds to i.e. correlates with a predisposition for high fat content of milk and high meat marbling.

Also in accordance with the invention the nucleotide polymorphisms are preferably located in a region which is responsible for the regulation of the expression of the product of the gene encoding DGAT.

More preferred the nucleotide polymorphisms which are analyzed by the method of the invention are single nucleotide polymorphisms (SNP).

In another preferred embodiment said testing in the method of the invention comprises hybridizing a herein above described nucleic acid molecule as a probe under stringent conditions to the nucleic acid molecules comprised in said sample and detecting hybridization. Such stringent conditions are known by a person skilled in the art and also described herein above.

More preferably said testing comprises digesting the product of said hybridization with a restriction endonuclease and analyzing the product of said digestion.

Even more preferred said probe is detectably labeled.

Alternatively, said testing comprises determining the nucleic acid sequence of at least a portion of said nucleic acid molecule. Methods for sequencing of nucleic acids are known in the art. An example for said testing for predisposition of individual animals by comparative sequencing is described herein below in example 6.

Preferably said determination of the nucleic acid sequence is effected by solid-phase minisequencing.

Also alternatively the testing further comprises, prior to analyzing the nucleic acid, amplification of at least a portion of said nucleic acid.

More preferred in said amplification reaction at least one of the primers employed in said amplification reaction is the primer or belongs to the primer pair as aforementioned, the method comprising assaying for an amplification product.

Even more preferred said amplification is effected by or said amplification is the polymerase chain reaction (PCR).

Furthermore, alternatively the method of the invention further comprises analyzing said nucleic acid by the use of:

(a) a primer extension assay;

(b) a differential hybridization assay; and/or

(c) an assay which detects allele-specific enzyme cleavage.

The underlying principles and the use of said assays has been described in an article of Asil Memisoglu (www.thebiotechclub.org/Tech/pharmacogenomics.html). Examples for said assays are known by a person skilled in the art. Furthermore, the method of analyzing said nucleic acid by the use of an assay which detects allele-specific enzyme cleavage is describe in example 8 herein below.

Furthermore, in an other embodiment the invention relates to a method of testing a mammal for its predisposition for fat content of milk and/or its predisposition for meat marbling, said method comprising the steps of:

(a) preparation of a tissue sample from the subject;

(b) contacting the sample with an aforementioned antibody specifically binding to an epitope of the polypeptide or fragment of SEQ ID NO: 2 the epitope comprising a alanine at position 232 but not to a polypeptide or a fragment of SEQ ID NO: 4 having a lysine at position 232 or specifically binding to an epitope of the polypeptide or fragment of SEQ ID NO: 4 the epitope comprising a lysine at position 232 but not to a polypeptide or a fragment of SEQ ID NO: 2 having a alanine at position 232; and

(c) detecting whether a specific binding of said antibody to its antigen has occurred.

Said method may comprise the transfer of the sample onto a membrane, e.g. by blot technique after electrophoresis. If so the detection whether a specific binding has occurred may comprise washing of the membrane to remove agent unspecifically bound to the membrane. Said detection may be performed by the use of agents which on the one hand are suitable for the detection of the presence of the specifically interacting agent. Furthermore said agents may comprises a domain or function which can be used for the generation of a detectable signal. The steps of contacting the proteins with said agents and detecting whether a specific interaction has occurred may be similar to the principle of immunodetection of proteins by Western Blot known to the person skilled in the art.

Preferably said method wherein the binding of the antibody which specifically binds to an epitope of the polypeptide or fragment of SEQ ID NO: 2 the epitope comprising a alanine at position 232 but not to a polypeptide or a fragment of SEQ ID NO: 4 having a lysine at position 232 indicates a predisposition of the mammal for low fat content of milk and to low meat marbling.

Also preferred, said method wherein the binding of the antibody which specifically binds to an epitope of the polypeptide or fragment of SEQ ID NO: 4 the epitope comprising a lysine at position 232 but not to a polypeptide or a fragment of SEQ ID NO: 2 having a alanine at position 232 indicates a predisposition of the mammal for high fat content of milk and to high meat marbling.

Also preferred is a method for testing of a mammal for its predisposition for low fat content and/or its predisposition for meat marbling comprising analyzing nucleotide positions 3343, 10433, 10434, 11030, 11048 and 11093 of the DGAT gene (SEQ ID NO:1), wherein the nucleotides CGCGCT, CGCGTT or GGCGTT at the above-indicated positions are indicative of low fat content of milk and low meat marbling.

Also preferred is a method for testing of a mammal for its predisposition for high fat content and/or its predisposition for meat marbling comprising analyzing nucleotide positions 3343, 10433, 10434, 11030, 11048 and 11093 of the DGAT gene (SEQ ID NO:1), wherein the nucleotides TMGCC, CAAGCC, CAAGCT, CAMCC or CAAACT at the above-indicated positions are indicative of high fat content of milk and high meat marbling.

More preferred the samples which are analyzed by the methods of the invention are isolated from cloven hoofed animals.

In a further more preferred embodiment said cloven hoofed animals are cattle, buffalos, yaks or pigs.

Finally the present invention relates in one embodiment to a kit comprising at least the aforementioned fragment, the aforementioned nucleic acid molecule, the aforementioned primer or primer pair, or one of the aforementioned in one or more containers.

The figures show

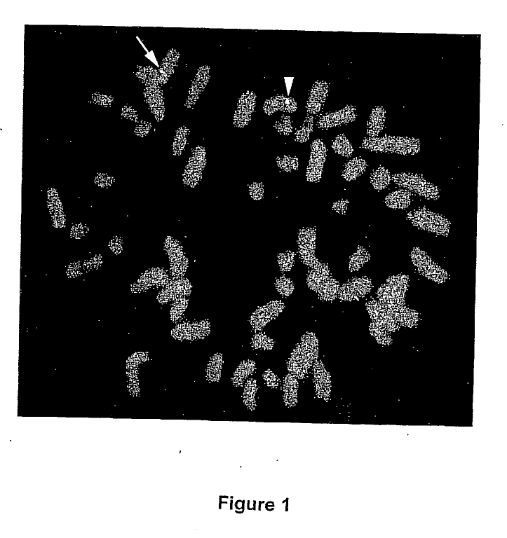

FIG. 1 Bovine metaphase spread after fluorescence in situ hybridization using BAG clone 56-F1. BAC-DNA was labeled with biotin using nick-translation. Detection of the hybridized probe was performed with streptavidin-Cy3. Photos were taken with a CCD-camera coupled to a Zeiss microscope with a magnification of 650×. The signals on both copies of chromosome 14 are indicated by arrow and arrow head. Note that one copy of chromosome 14 (signal indicated by arrow) is involved in a Robertsian fusion with chromosome 20.

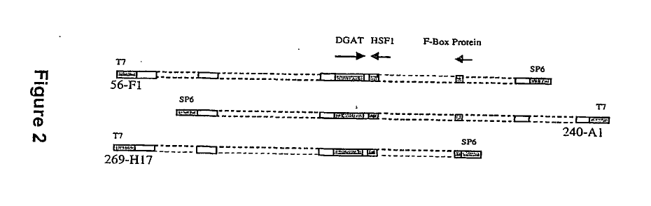

FIG. 2 Partial maps of three BACs (56-F1, 240-A1, 269-H17). Solid lines represent sequenced parts. The vector sequences are shown as gray boxes. T7 and SP6 refer to the primers used for BAG-end sequencing. The colored boxes represent genes: DGAT, diacylglycerol acyltransferase; HSF1, heat shock transcription factor 1; FPXL6, f-box and leucine-rich repeat protein 6. Annotation of the sequences is based on a high similarity with the corresponding human sequences. The arrows indicate the orientation of the genes. Drawings are not to scale.

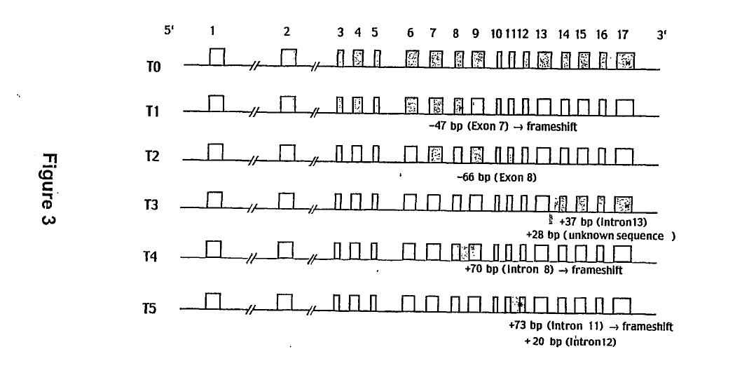

FIG. 3 EST-derived transcript map of the bovine DGAT gene. The blue areas represent sequences covered by the ESTs. T0 is composed of ESTs AW483961, AW486026, AW652329, BE664362, BE753833, BE664357, T1 of AW446908, T2 of AW446985, T4 of AW326076 and T5 of BE486748. The approximate position of stop codons are indicated by asterisks. T1 and T2 may represent alternative transcripts, with T1 leading to a truncated gene product. T3 contains 28 bp that are not found in the genomic sequence and therefore most likely are artefacts. T4 and T5 probably represent unprocessed transcripts.

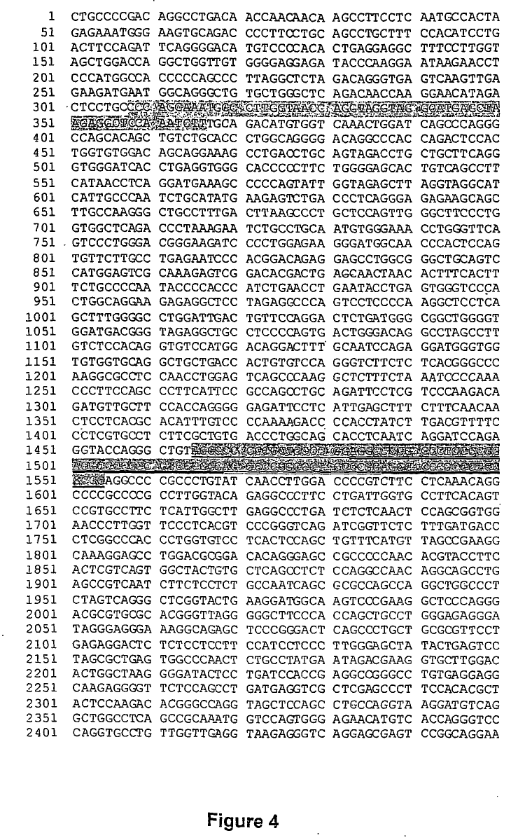

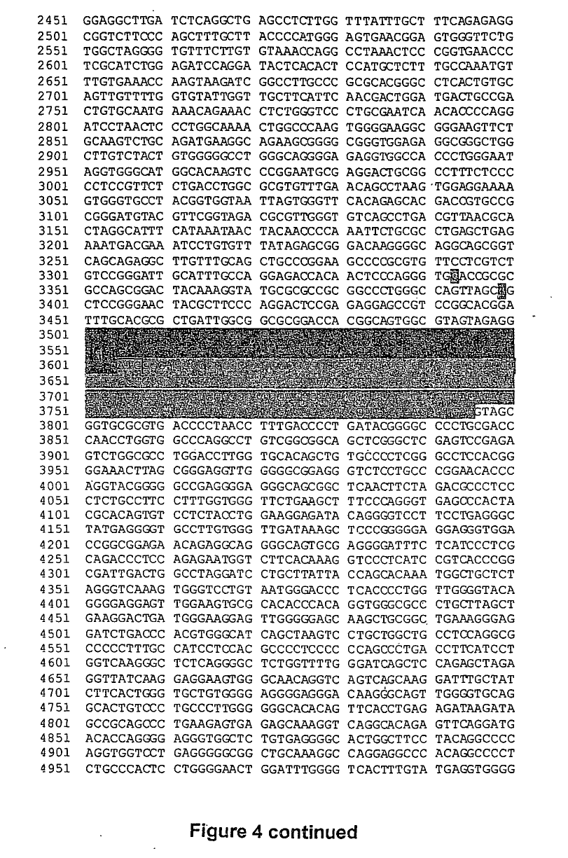

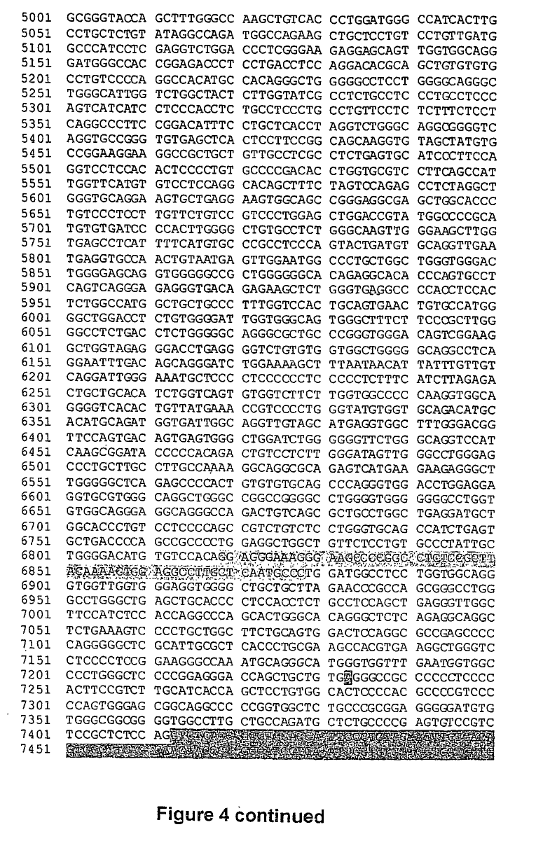

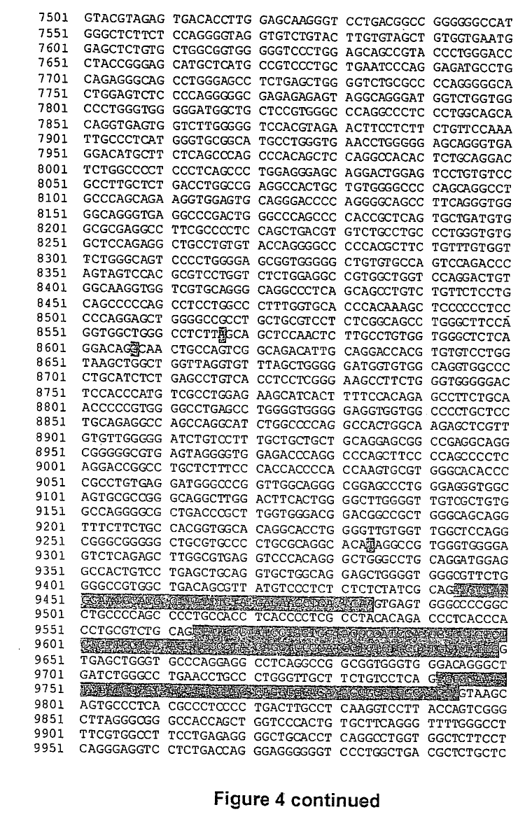

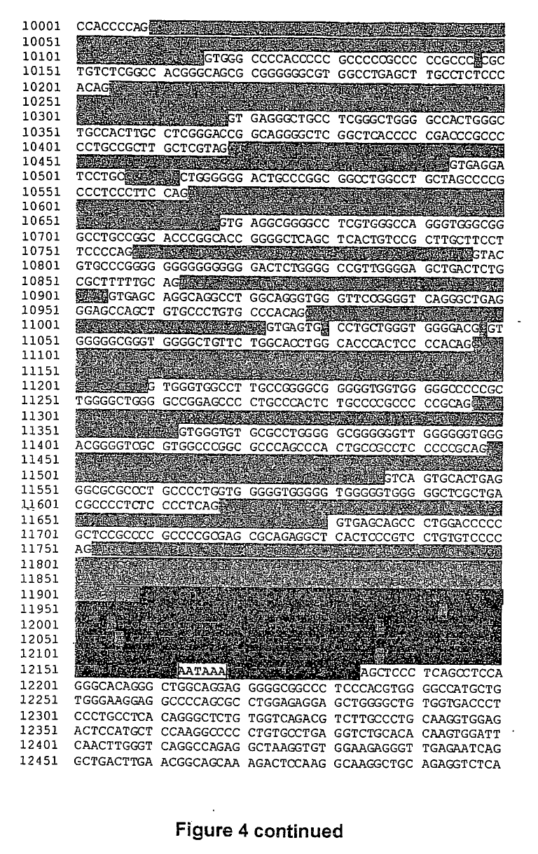

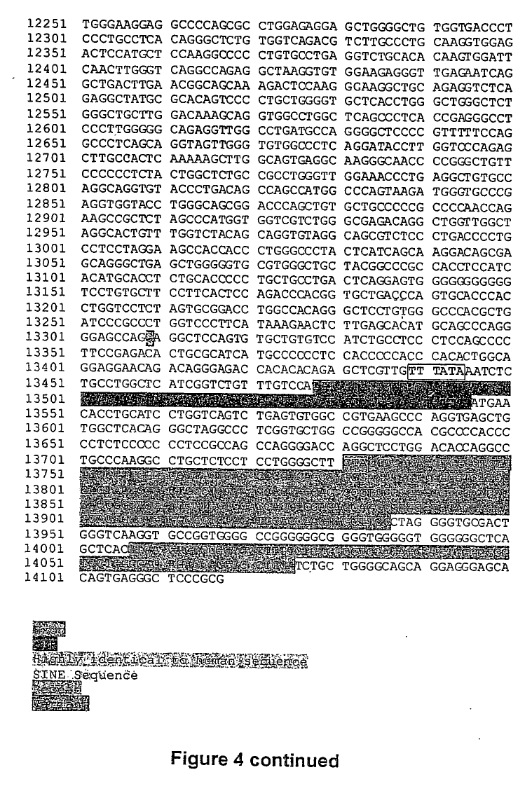

FIG. 4 Bovine genomic sequence containing DGAT and parts of HSF1 (3'end). Start codon (position3605), stop codon (position 11906) and polyA signal (position 12163) of DGAT and stop codon (position 13731) and putative polyA signal (position 13439) of HSF1 are in bold.

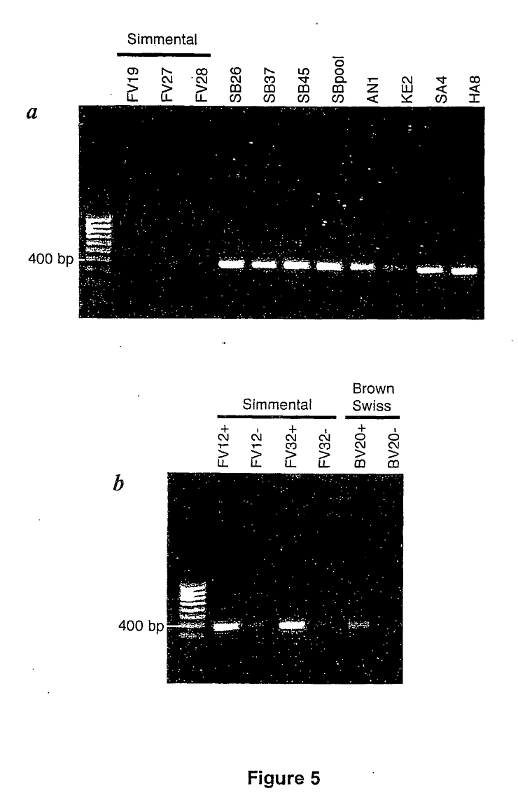

FIG. 5 Variable PCR amplification by a, individual animals and b, pooled samples.

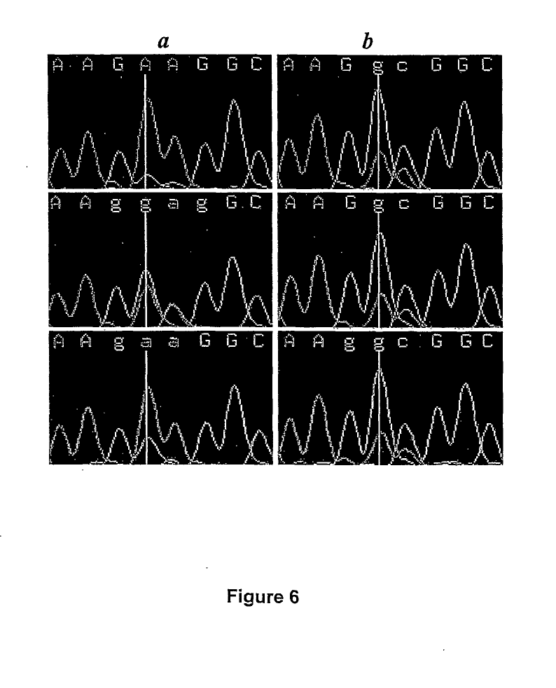

FIG. 6 Consed views of sequencing traces for positions 10430-10437 within DGAT demonstrating the effect of DMSO in the PCR at variable positions 14433 and 14434 of a heterozygous animal (GC/AA). a, three repetitions without DMSO. b, three repetitions with 5% DMSO. Average normalized amplitude values (± standard deviation) in a: A 1.06±0.25, A 0.61±0.16, G 0.56±0.31, C 0.21±0.14; in b: A 0.42±0.02, A 0.22±0.02, G 1.38±0.02 C 0.59±0.03.

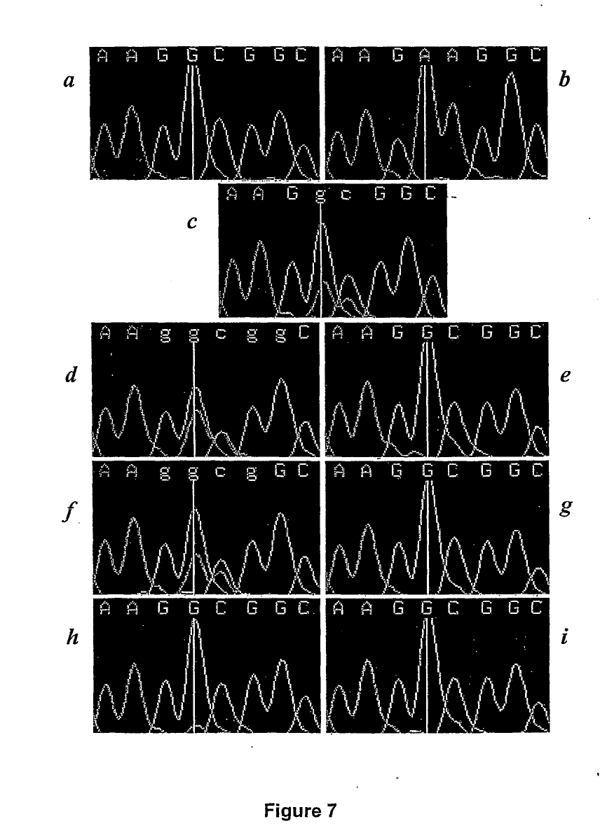

FIG. 7 Consed views of sequencing traces for positions 10430-10437 within the DGAT coding sequence. Positions 10433 and 10434 are variable. (a), (b) represent homozygous animals (GC/GC, AA/AA), respectively) and (c) a heterozygous animal (AA/GC). (d) and (e) show the frequency shift between the pools FVpool12+ (breeding value milk fat % (BVMF)=+0.729±0.045) and FVpool12− (BVMF=−0.445±0.042), (f) and (g) between pools FVpool32+(BVMF=+0.669±0.063) and FVpool32− (BVMF=−0.381±0.059), (h) and (i) between pools BVpool20+(BVMV=+0.421±0.113) and BVpool20−(BVMF=−0.305±0.057).

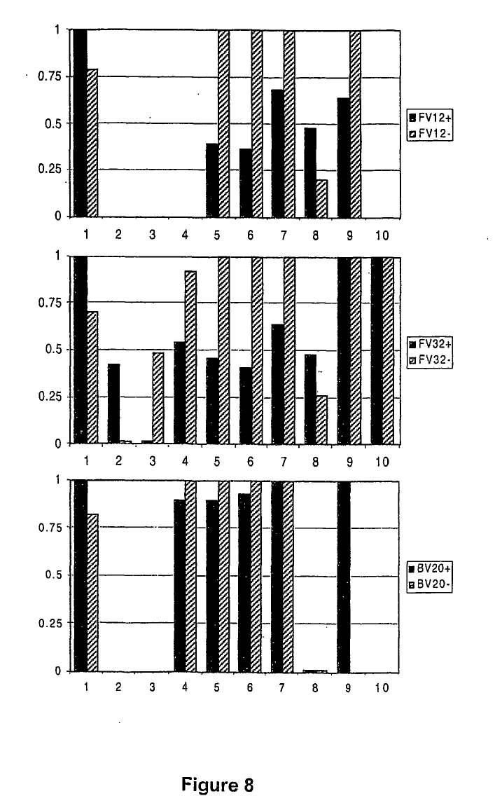

FIG. 8 Allelic frequencies in pooled samples from animals with high (FV12+, FV32+, BV20+) and low (FV12−, FV32−, BV20−) breeding values for milk fat content at variable positions in and around DGAT The numbers below the x-axis refer to the following positions (according to the numbering in FIG. 3): 1, 3343; 2, 8567; 3, 8607; 4, 9284; 5, 10433; 6, 10434; 7, 11030; 8, 11048; 9, 11993; 10, 130309. The variable positions 5 and 6 are responsible for the K232A substitution, with the frequency of the A-encoding allele being indicated.

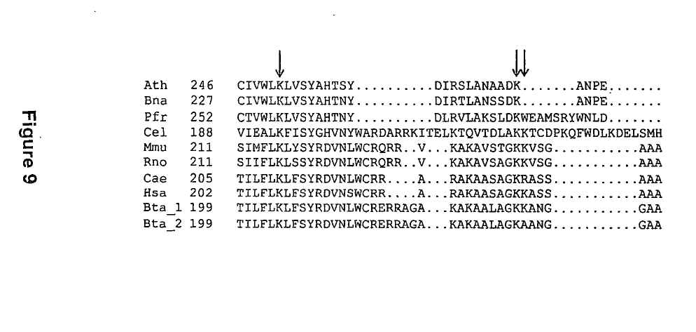

FIG. 9 Alignment of the DGAT amino acid sequences of Arabidopsis thaliana (Ath), Brassica napus (Bna), Perilla fructescens (Pfr), Caenorhabditis elegans (Cel), Mus musculus (Mmu), Rattus norvegicus(Rno), Ceropithecus aethiops (Cea), Homo sapiens (Hsa) and two alleles of Bos taurus (Bta_{—}1, Bta_{—}2) using PILEUP of the GCG package. Sequences are assembled using BOXSHADE (http://www.isrec.isb-sib.ch:8080/software/BOX_form.html). Numbers on the left indicate amino acid positions. Red letters indicate identical amino acids. Blue letters indicate conserved amino acids. The red arrows indicate identical lysine residues that might play a role in Acyl CoA binding. The blue arrow indicates conserved amino acids in animal species and in the bovine allele associated with high milk fat content. The lysine to alanine mutation at this position is not conservative. The alanine residue of the allele associated with low milk fat content could have a negative effect on the Acyl CoA binding capacity of DGAT.

FIG. 10 Hydrophobicity plot of DGAT as assessed by Kyte-Doolittle analysis (http://bioinformatics.weizmann.ac.il/hydroph/plot_hydroph.html). Hydrophobic regions are above the horizontal line. a Translated transcript T0 (The effect of the K232A substitution is indicated in red (K, blue; A, red)). b Translated transcript T2 (missing amino acids 230 to 251 of transcript T0).

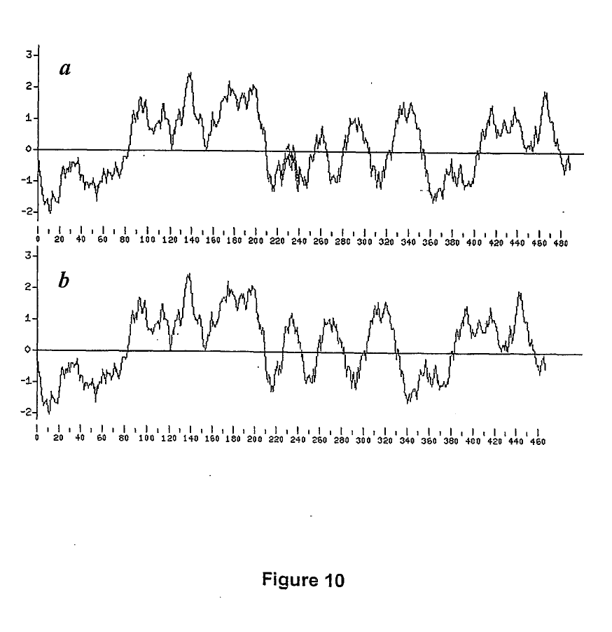

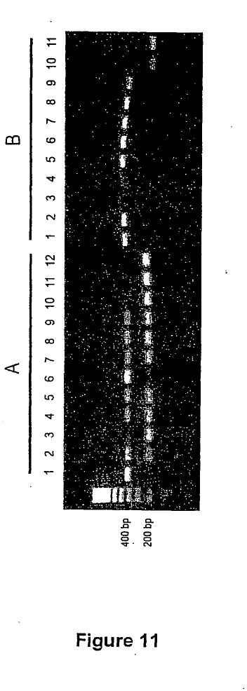

FIG. 11 Detection of the allelic variation at the nucleotide positions 10433 and 10434 of the DGAT gene by Cfrl-cleavage in a 411 bp PCR product from bovine genomic DNA (primers 1532 and 1636). Cleavage by Cfrl is diagnostic for the alanine bearing allele. Panel A, 5% DMSO in PCR reaction; panel B, PCR without DMSO. Panel A, lane 1, lane 6: homozygous for lysine variant; Panel A, lane 2, 4, 5, 7, 8, 9: heterozygous; Panel A, lane 3, 10, 11, 12: homozygous for alanine variant. Panel B, lanes 1-11 represent the same animals as lanes 1-11 in panel A. Preferential amplification of the lysine variant (nucleotides AA) over the alanine variant (nucleotides GC) prevents the detection of the alanine variant in the heterozygotes.

FIG. 12 Haplotypes of DGAT1 based on nucleotide positions 3343, 10433, 10434, 11030, 11048, 11993 determined by direct sequencing (A) and preliminary frequency estimates for the lysine (dark) and alanine (light) encoding alleles determined by RFLP assay (B). Anatolian Black is a breed indigenous of a region known as the site of domestication of the European Bos taurus [Medjugorac, 1994].

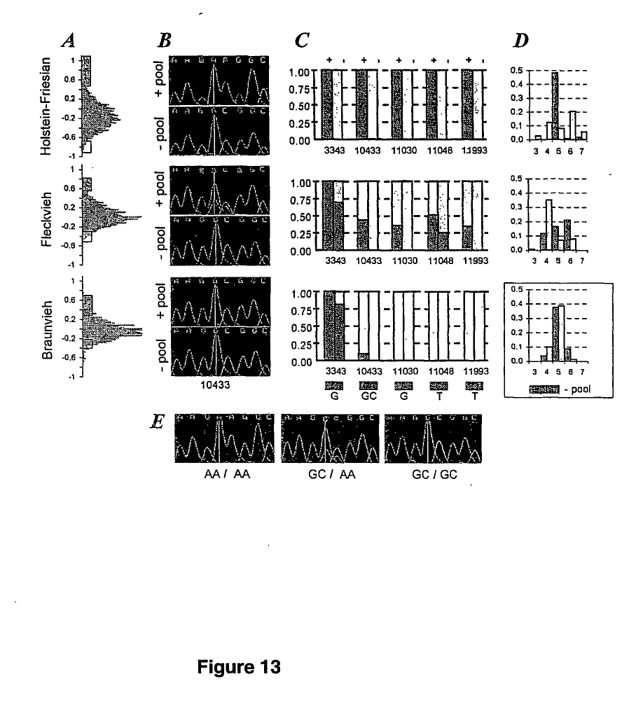

FIG. 13(A) Distributions of breeding values for milk fat content of Holstein-Friesian (HF), Fleckvieh (FV) and Braunvieh (BV) artificial insemination (A1) bulls born in 1990 or later. Colored areas indicate the range of the breeding values, from which bulls were chosen for the extreme positive (+, dark) and negative (−, light) pools for HF (32 per pool), FV (32 per pool) and BV (20 per pool), respectively. HF bulls were selected among 2857 AI bulls. The mean breeding value for milk fat content of the unselected bulls was −0.148, the standard deviation was 0.284. Bulls with breeding values above 0.48 and below −0.68 were selected. The mean breeding values (± standard deviations) of pooled groups were as follows: HF32+, 0.622±0.125; HF32−, −0.771±0.063. FV bulls were selected among 4070 AI bulls. The mean breeding value for milk fat content of the unselected bulls was 0.089, the standard deviation was 0.217. Bulls with breeding values above 0.5 and below −0.3 were selected. The mean breeding values (± standard deviations) of pooled groups were as follows: FV32+, 0.683±0.153; FV32−, −0.454±0.061. BV bulls were selected among 656 AI bulls. The mean breeding value for milk fat content of unselected bulls was 0.006, standard deviation 0.185. Bulls with breeding values above 0.2 and below −0.2 were selected. Mean breeding values (±standard deviations) of pooled groups were as follows: BV20+, 0.424±0.156; BV20-, −0.317±0.096. (B, E) Consed views of sequencing traces for positions 10430-10437 within the DGAT1 coding sequence for individual animals (E) and DNA pools (B). (C) Allele frequency shifts. Position of variant and bases are indicated below horizontal axis. Frequencies at position 10433 are determined by genotyping individual animals by sequencing or RFLP assay. Frequencies at position 11030 and 11048 in FV+pool are determined by sequencing. The other frequencies represent estimates from sequence traces (as described in methods). Variable positions 10433 and 10434 are responsible for the K232A substitution. (D) Bars represent the frequencies of alleles with 3, 4 5, 6 and 7 repeat units in 5′-region of DGAT1 in +pool (dark) and—pool (light) for each breed.

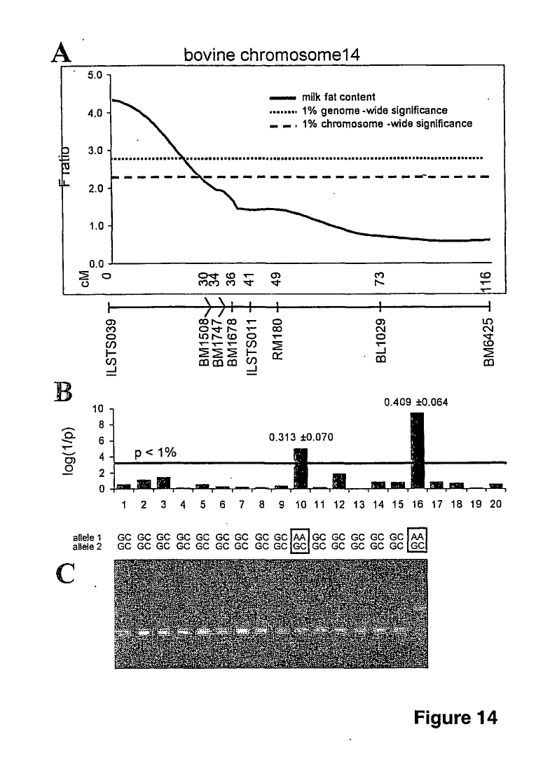

FIG. 14(A) Across family test statistic curve for QTL analyses of milk fat content on chromosome 14 for a Fleckvieh granddaughter design. F ratios testing for the presence of a segregating QTL are plotted for given positions along the chromosome. The marker map with distances in cM between markers is shown on the x-axis. Empirical chromosome-wide and genome-wide 1% significance levels achieved via 10,000 permutations are indicated as horizontal lines. (B) The bars show transformed significance levels (log (1/p)) of the test statistic for a segregating QTL present within each family (x-axis). The horizontal line indicates the transformed 1% significance level for a single family after correcting for multiple testing of 20 families. QTL-effects for milk fat content and their respective standard errors are shown on top of the bars for significantly segregating sires. (C) Detection of allelic variation at nucleotide positions 10433 and 10434 (K232A) of the DGAT1 gene by Cfrl-cleavage in a 411 bp PCR product from bovine genomic DNA of sire 1 to 16. Cleavage by Cfrl is diagnostic for the allele encoding alanine (GC). No DNA samples were available for sires 17 to 20.

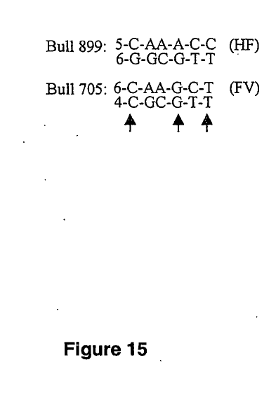

FIG. 15 Haplotypes of two segregating (Qq) bulls. HF: Holstein-Friesian, FV: Fleckvieh. The arrows indicate the homozygous sites, implicating these variants are not causal.

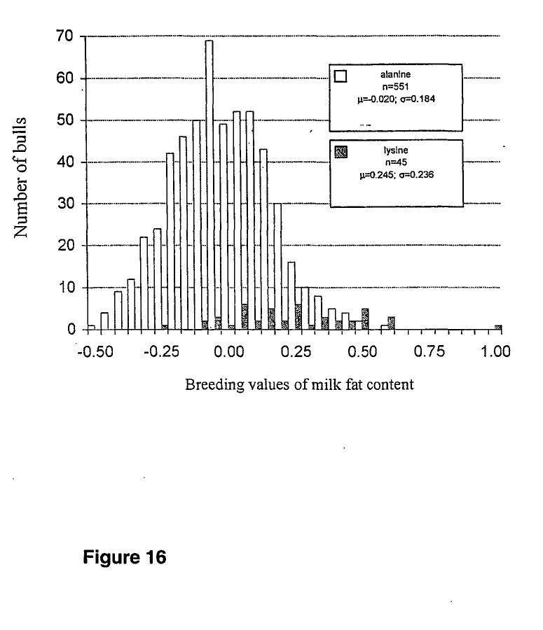

FIG. 16 Distribution of breeding values of sons of non segregating sires according to whether or not they have received the lysine alleles from their dams.

The examples illustrate the invention:

### EXAMPLE 1

### Preparation of the Primers

All primers used in the following procedures were designed using the Primer3 program (www.genome.wi.mit.edu/cgi-bin/primer/primer3_www.cgi). Unless indicated directly in the text, primer sequences are listed in Table 1 and Table 2.

### EXAMPLE 2

### Radiation Hybrid Panel Mapping

25 ng of genomic DNA from the human-hamster radiation hybrid panel Genbrige 4 (HGMP Resource Center) were amplified with one set of primers specific for the human DGAT gene (forward (1534), 5′-GAGGCCTCTCTGCCCTATG-3′; reverse (1538), 5′-TTTATTGACACCCTCGGACC-3′). PCR was performed on 84 clones of the RH-panel and analyzed by gel electrophoresis (2% agarose). PCR conditions were as follows: 10 μl total volume containing 0.5 μM of each Primer, 200 μM of each dNTP, 1 μl 10×PCR reaction puffer, 1.5 mM MgCl₂ and 0.5 U AmpliTaq polymerase (PE Biosystems). The reactions were amplified in a TGradient Thermocycler (Biometra) under following conditions: 1 cycle at 94° C. for 3 min, followed by 30 cycles at 95° C. for 30 sec, 60° C. for 1 min, 72° C. for 1 min, followed by 1 cycle at 72° C. for 10 min. Positive and negative PCR assays were reported as 1 and 0, respectively, unclear assays as 2. The data were analyzed with a program provided from The Sanger Center (www.sanger.ac.uk/Software/RHserver/RHserver.shtml).

### EXAMPLE 3

### Screening of Bovine BAC Library

Screening was performed by hybridization of high-density filters. A specific PCR product of 565 bp (forward primer (1599), 5′-CGAGTACCTGGTGAGCATCC-3′; reverse primer (1601), 5′-TGTGCACAGCACTTTATTGAC-3′) was used as a probe for radioactive screening of the bovine RPCl-41 genomic BAC library (Warren et al., 2000). PCR conditions were as follows: 20 μl total volume containing 0.5 μM of each Primer, 200 μM of each dNTP, 2 μl 10×PCR reaction puffer, 1.5 mM MgCl₂ and 1.0 U AmpliTaq polymerase (PE Biosystems). Temperature cycling were as follows: 1 cycle at 94° C. for 3 min, followed by 30 cycles at 95° C. for 30 sec, 60° C. for 1 min, 72° C. for 1 min, followed by 1 cycle at 72° C. for 10 min. Probes were labeled with 50 μCi of alpha[³²P]dATP using the Megaprime DNA labeling system following the manufacturer protocol (Amersham). Labeled probe was added to the filter in Church buffer and hybridized at 67° C. overnight. Filters were washed twice in 2×SSC and once in 0.5×SSC+1% SDS for 20 minutes at 63° C., respectively. Filters were exposed to Fuji NewRX film at −80° C. for 5 h. Positive clones were confirmed by PCR amplification (same primer and conditions as above) and DNA sequencing.

### EXAMPLE 4

### Sequencing from BAC-DNA

BAC-DNA was isolated using the QIAGEN Large-Construct Kit (Qiagen) following the manufacturer protocol. In the first step, primers (Table 1) for genomic walking were derived from the known bovine sequence of exon 2 (forward, 1602) and exon 3 (reverse, 1634). In addition to that, a primer (forward, 1632) was derived from the human sequence of exon 1 showing high homology to Cercopithecus aethiops (accession#: AF236018), Mus musculus (accession#: NM_{—}010046), Rattus nor-vegicus (accession#: AF296131). Further primers were derived from the obtained sequences. Conditions of sequencing reaction were as follows: 150 ng BAC-DNA, 0.4 mM primer and 10 μl BigDye Ready Reaction Mix (PE Biosystems) were combined in a total volume of 25 μl. Temperature cycling were as follows: 1 cycle at 96° C. for 5 min, followed by 80 cycles at 96° C. for 20 sec, 55° C. for 10 sec, 60° C. for 4 min. DNA was precipitated with 60% isopropanol, washed with 75% isopropanol, loaded on a 36 cm WTR acrylamid gel (5.5%) on an ABI Prism 377 DNA sequencer. Sequence data were analyzed using the Phred/Phrap/Polyphred/Consed software suite (Nickerson et al., 1997; Ewing and Green, 1998; Ewing et al., 1998; Gordon et al., 1998).

### EXAMPLE 5

### Preparing of Genomic DNA Samples

DNA was prepared from bull semen. After washing with TE buffer (10 mM Tris HCl, 1 mM EDTA), cells were lysed by adding 500 μl PK buffer (20 mM Tris HCl, 4 mM EDTA, 10 mM NaCl), 100 μl SDS (10%), 25 μl DTT (1 M), 60 μl proteinase K (20 mg/ml) and incubated at 50° C. overnight. Phenol/chloroform extraction was carried out in 9.5 ml VACUTAINER® tubes (#366510, Becton Dickinson). In the first step 800 μl of phenol/chloroform/isoamylalcohol (25:24:1) was added, mixed thoroughly and centrifuged for 15 min at 2000 g at RT. Traces of phenol were removed by centrifugation after adding 800 μl of chloroform/isoamylalcohol (24:1). DNA was precipitated with ethanol and resuspended in TE buffer. DNA concentration was measured using a fluorometer and adjusted to a concentration of 25 ng/μl. Quality and quantity of DNA was indepently assessed through agarose gel electrophoresis and by performing PCR (primer and conditions as in Screening of bovine BAC library). Only DNA samples showing perfect results in both gel electrophoresis and PCR were used for DNA samples for individual animals and for composing pooled DNA samples.

### EXAMPLE 6

### Comparative Sequencing

Screening for variations was performed using the DNA samples of the individual animals and the pooled DNA samples in combination with several primer sets (Table 2). Each DNA sample (50 ng) was amplified in 20 μl reactions containing 0.5 μM of each Primer, 200 μM of each dNTP, 1 μl 10×PCR reaction puffer (containing 15 mM MgCl₂), 0.5 U HotStar polymerase (Qiagen). Temperature cycles were as follows: 1 cycle at 95° C. for 15 min, followed by 35 cycles at 94° C. for 1 min, 60° C. for 1 min, 72° C. for 1 min, followed by 1 cycle at 72° C. for 10 min. The PCR amplified fragments were directly purified with the QIAquick PCR purification kit (Qiagen) and analyzed on a 1.5% agarose gel. Conditions of sequencing reaction were as follows: In a total volume of 10 μl was combined 20 ng PCR fragment, 0.5 μM Primer, 4 μl BigDye Ready Reaction Mix (PE Biosystems). Temperature cycling were as follows: 1 cycle at 96° C. for 15 sec, followed by 25 cycles at 96° C. for 10 sec, 51° C. for 5 sec, 60° C. for 4 min. DNA was precipitated in 60% isopropanol, washed with 75% isopropanol and run on a 36 cm WTR 5.5% acrylamid gel on an ABI Prism 377 DNA sequencer. Sequence data were analyzed using the Phred/Phrap/Polyphred/Consed software suite (Nickerson et al., 1997; Ewing and Green, 1998; Ewing et al., 1998; Gordon et al., 1998).

### EXAMPLE 7

### Estimation of Allelic Frequencies Based on Sequencing Traces

The amplitude values at the variable positions were extracted from data files “.poly” created by the base calling program phred. The amplitude value for a given base was divided by the normalization factor for that base. The normalized amplitude value of pooled DNA (P) was compared with the amplitude value of homozygous (Ho) or heterozygous (He) individual animals or monomorphic pools. Averages were taken when amplitude values were available for more than one animal. Frequency estimates (F) were obtained by the following calculations: F=P/Ho or F=(0.5×P)/He.

### EXAMPLE 8

### RFLP-Analysis of PCR-Fragments

The genotyp of an individual or group of animals was tested by the use of RFLP-analysis. Detection of allelic variation at the nucleotide positions 10433 and 10434 of the DGAT gene was effected by CfrI-cleavage in a 411 bp PCR product from bovine genomic DNA (primers 1532 and 1636). Cleavage by Cfrl is diagnostic for the alanine bearing allele. The result of a test is shown in FIG. 11. PCR reactions were carried out in the presence (panel A) or absence (panel B) of 5% DMSO. PCR-products were isolated following common protocols as known by a person skilled in the art and incubated with the restriction endonuclease CfrI under conditions in line with manufactures advice. FIG. 11 shows in panel A, lane 1 and lane 6 samples, which are homozygous for lysine variant. In lane 2, 4, 5, 7, 8, 9 of panel A samples with heterozygous genotype are shown. Furthermore, lane 3, 10, 11, 12: show samples which are homozygous for alanine variant. In panel B, lanes 1-11 samples of the same animals as shown in lanes 1-11 of panel A are displayed. Preferential amplification of the lysine variant (nucleotides AA) over the alanine variant (nucleotides GC) prevents the detection of the alanine variant in the heterozygotes.

### EXAMPLE 9

### Direct Sequencing Reveals at least 8 Haplotypes of DGAT1

Direct sequencing in animals belonging to different breeds of Bos taurus taurus and Bos taurus indicus as well as in animals of Bos grunniens (yak) and Bubalus bubalus (water buffalo) at 6 of the variable nucleotide positions allowed to derive at least 8 haplotypes (FIG. 12). Lysine encoding haplotypes are present in yak and water buffalo. Thus, the lysine encoding variant is likely to represent the ancestral state of DGAT1. However, the K232A substitution is likely to have taken place early in the history of domesticated cattle or even before domestication as surmised by the presence of the alanine variant in the “old” cattle breed Anatolian Black. An RFLP assay was applied to obtain preliminary estimates on the frequency of the lysine and alanine encoding alleles in several cattle breeds and species of Bovinae subfamily (FIG. 12).

### EXAMPLE 10

### Distribution of Breeding Values for Milk Fat Content

The frequencies at 6 variable positions in the pools of animals with high and low breeding values for milk fat content, respectively, are visualized in FIG. 13. There are distinct differences for the Fleckvieh and Holstein-Friesian-Friesian breeds in the frequencies between the groups of animals with low and high breeding values for milk fat content, respectively, indicating association between variation in the DGAT1 gene and genetic variation of the milk fat content. The most extreme differences are between the “low” and “high” pools in the Holstein-Friesian breed. In both breeds, the lysine encoding variant is more frequent in animals with high breeding values for milk fat content. The lysine encoding allele is also slightly less more frequent in the Braunvieh animals from the high end of the distribution of the milk fat content breeding values.

### EXAMPLE 11

### Across Family Test Statistic Curve for QTL Analyses of Milk Fat Content on Chromosome 14 for a Fleckvieh Granddaughter Design

Another argument for DGAT1 (or linked loci) being responsible for the QTL-variation on chromosome 14 is provided by the results obtained from interval QTL mapping in the Fleckvieh breed using a half-sib design, the so called granddaughter design. The test statistic for the presence of a QTL along chromosome 14 (FIG. 14) indicates the most likely position of the QTL close to marker ILSTS039. Evidence was highly significant for segregation of the QTL in two out of 20 families (FIG. 14). Estimates of QTL effects for milk fat content in the segregating families were found to be 0.313±0.070 and 0.409±0.064, respectively. These effects greatly exceed the genetic standard deviaion of 0.2 in the Fleckvieh population. The genotypes at the predicted K232A substitution determined by an RFLP assay are compatible with the heterozygous status of the segregating (Qq) sires and homozygosity of the alanine encoding variant of the non-segregating (most likely qq) sires (FIG. 14).

### EXAMPLE 12

### Haplotypes of Two Segregating (Qq) Bulls

Direct sequencing of DGAT1 from DNA and determining the repeat number of the 5′-VNTR in the two segregating bulls and some of their progeny allowed to derive the haplotypes based on the genotypes of the homozygous progeny. The lysine encoding variant is present on two different haplotypes, i.e. the only lysine bearing haplotype in Holstein-Friesian and a Fleckvieh-specific haplotype (FIG. 12, FIG. 15). This could indicate that a lysine encoding allele has been introduced into Fleckvieh from Holstein-Friesian. Pedigree analysis indeed shows that the great-grandfather of bull 899 was a purebred Holstein-Friesian sire while there is no indication of Holstein-Friesian ancestry for bull 705. Three of the 7 variable positions that make up the haplotypes are homozygous in Qq bull 705 (FIG. 15). Thus they can be excluded to be causal. The variants responsible for the K232A polymorphism, however, are heterozygous in both Qq bulls.

### EXAMPLE 13

### Distribution of Breeding Values of Sons of Non Segregating Sires

An independent association study was carried out based on the breeding values for milk fat content of the sons of non segregating sires. These sons were grouped according to the allelic variant (lysine or alanine) which they have received from their dams as determined by the RFLP assay. The respective means of breeding values were compared after correction of half the sire's breeding value (FIG. 16). The difference of +0.265 for the group carrying the lysine variant was highly significant (P<0.0001) and strongly supports the size of the gene substitution effect found via linkage analysis. It is also in agreement with the results of the association study presented above. Since the dams can be considered to represent a random sample of the Fleckvieh population with regard to milk fat content, the association involving the sons of non segregating sires is not likely to be confounded by admixture.

### EXAMPLE 14

### Mast Experiment “Dummersdorf”—Evaluation of DGAT

Objective: Impact of DGAT for Intramuscular Fat Content.

Material:

The experiment is based on data obtained from 56 slaughtered fattened animals of both gender of the races Deutsche Holstein Friesian (n=29) and Charolais (n=27). IMF-values of MLD (IMF_MLD) and Bratenstück [bitte übersetzen] (IMF_SEMI) and the exchange of K232A in DGAT were determined. The allelic frequency of the lysine variant, in both tested samples, were estimated as 11% for Charolais and 45% for Holstein Friesian.

Statistical Analysis:

The statistical analysis was established by using the method of least squares which is part of the program SAS (Version 8.02). The analysis of the total material was based on the model:

Yᵢⱼₖₗₘ=Raceᵢ+Fatherⱼ(Raceᵢ)+Genderₖ+DGAT−Genotypeₗ+eᵢⱼₖₗₘ

In another analysis, the data was evaluated for each race separately, wherein the effect of the race of the above-indicated model is left out. By employing the variance analysis, the contribution of the individual factors for the establishment of the IMF properties was tested. Moreover, least square means were calculated for the specific genotypes, the differences of which represent an estimate reflecting the differences between these genotypes.

Results:

All experiments showed a significant gender-impact. Table 13 summarizes the F- and p-values and levels of significance (n.s: not significant; *: p<0.05) of the variance analysis for the effect of DGAT genotypes. The results indicate a significant impact of DGAT on IMF_SEMI and no indication of an impact on IMF_MLD. The increased F-values of Holstein Frisian in comparison with Charolais (when data was evaluated for each race separately) may rest on the fact that a homozygous lysine variant never occurred in Charolais. From analyses on the TG locus a recessive inheritance is suggested, wherein Alanin is dominant over Lysine, thus, preventing the detection of the effect on IMF in Charolais.

Table 14 summarizes the least square means and their standard error. The predominance of UL genotypes over L/A and A/A, as evident from the analysis, amounted to 1.6% percent in IMF_SEMI. When analyzed separately, on average a similar difference is found in Holstein Frisian. However in the latter case, the results for the genotypes L/A and A/A are less uniform and have to be discussed with caution since they are associated with a high standard error. The differences observed are of a magnitude which are likely to be only possible in extremely fastened animals. The resulting high variability of starting material may also be the reason for a lack of statistical support of the large differences in IMD_MLD of Hostein Friesian.

### Tables

TABLE 1

Tables:
Primers used for sequencing of BAC-DNA

| Location | | | |
|---|---|---|---|
| in DGAT | # | Direction | Sequence |
| 5′end | 1738 | reverse | 5′-TGATGCCTACCTAAGCTCTACC- |
| | | | 3′ |
| 5′end | 1739 | reverse | 5′-TTTAGGGTCTGAGCCACCAG-3′ |
| 5′end | 1728 | reverse | 5′-TCCCGACTCTTTGTGACTCC-3′ |
| 5′end | 1734 | reverse | 5′-TGGATTGCAAAGTCCTGTCC-3′ |
| 5′end | 1717 | reverse | 5′-CAGGAAGGGCCTCTGTACC-3′ |
| 5′end | 1716 | reverse | 5′-ACAGCTGGAGTGAGGACACC-3′ |
| 5′end | 1710 | reverse | 5′-CCCTCAGCGCTAGGACTC-3′ |
| 5′end | 1709 | reverse | 5′-TGTCTTGGAGTAGCGTGTGG-3′ |
| 5′end | 1706 | reverse | 5′-AGGCCCCCACAGTAGACAAG-3′ |
| 5′end | 1705 | reverse | 5′-ACGGTCGTGCTCTGTGAAC-3′ |
| 5′end | 1699 | reverse | 5′-CCCTTGTCCCGCTCTATAAAC-3′ |
| 5′end | 1698 | reverse | 5′-CGCGCATACCTTTGTAGTCC-3′ |
| 5′end0 | 1697 | reverse | 5′-CGCCTCTACTACGCCACTG-3′ |
| Exon 1 | 1632 | forward | 5′-GCCACTGGGAGCTGAGG-3′ |
| Intron 1 | 1681 | reverse | 5′-ACAGCTGTGCACCAAGGTC-3′ |
| Intron 1 | 1680 | forward | 5′-TGGCTGCTCTAGGGTCAAAG-3′ |
| Intron 1 | 1693 | forward | 5′-ATCTTCACTGGGTGCTGTGG-3′ |
| Intron 1 | 1694 | forward | 5′-CTGCTCCTGTCCTGTTGATG |
| Intron 1 | 1696 | reverse | 5′-AGCCACCTCATGCTACAACC-3′ |
| Intron 1 | 1695 | reverse | 5′-GCCCTCTTCTTCATGACTCTG-3′ |
| Intron 1 | 1679 | reverse | 5′-GGCCACCATTCAAACCAC-3′ |
| Exon 2 | 1602 | forward | 5′-GAATTGGTGTGTGGTGATGC-3′ |
| Intron 2 | 1675 | reverse | 5′-GGTAGGGTCCCAGGGTACG-3′ |
| Intron 2 | 1673 | forward | 5′-GCCACACTCTGCAGGACTC-3′ |
| Intron 2 | 1674 | reverse | 5′-CAGTCCTGCTCCCTCCAG-3′ |
| Intron 2 | 1671 | reverse | 5′-TGACAGGCTCAGAGATGCAG-3′ |
| Intron 2 | 1660 | reverse | 5′-AGCCCCAGTGAAGTCCAAG-3′ |
| Exon 3 | 1634 | reverse | 5′-TAGAAATAACCGTGCGTTGC-3′ |
| Exon 4 | 1633 | reverse | 5′-ACCTGGATGGGGTCCAC-3′ |
| 3′end | 1593 | forward | 5′-GTGGGTGTTGGACTGCTTTG-3′ |
| 3′end | 1711 | forward | 5′-CCATGCTCTGGAAACCCTAC-3′ |
| 3′end | 1729 | forward | 5′-TCAGCAGGTAGTTGGGTGTG-3′ |
| 3′end | 1730 | forward | 5′-GAAACCCTGAGGCTGTGC-3′ |
| 3′end | 1732 | forward | 5′-CCCACCTGGTCCTCTAGTGC-3′ |
| 3′end | 1733 | forward | 5′-CCAGGAGGCTCCAGTGTG-3′ |
| 3′end | 1737 | forward | 5′-GTTCTGAGCCCGTCAGCAG-3′ |
| 3′end | 1739 | forward | 5′-TTTAGGGTCTGAGCCACCAG-3′ |

TABLE 2
Primers used for PCR and comparative sequencing of genomic DNA

| Location in | Forward primer | | Reverse primer | |
|---|---|---|---|---|
| DGAT | # | Sequence | # | Sequence |

| | | | | |
|---|---|---|---|---|
| Exon 1 | 1701 | 5′-CGCGTTGGGTGTCAGC-3′ | 1681 | 5′-ACAGCTGTGCACCAAGGTC-3′ |
| Exon 2 | 1702 | 5′-TGGCTTCTGCAGTGGACTC-3′ | 1675 | 5′-GGTAGGGTCCCAGGGTACG-3′ |
| Exon 3-4 | 1670 | 5′-GTGGCTGACAGCGTTATGTC-3′ | 1676 | 5′-GTTCAGGCCCAGATCAGC-3′ |
| Exon 4-6 | 1614 | 5′-TATGGCATCCTGGTGGAC-3′ | 1617 | 5′-AGTGATAGACTCGAGGAGAAAGG-3′ |
| Exon 6-7 | 1616 | 5′-GGAGCTCTGACGGAGCAG-3′ | 1635 | 5′-GTTGACGTCCCGGTAGGAG-3′ |
| Exon 7-9 | 1532 | 5′-GCACCATCCTCTTCCTCAAG-3′ | 1636 | 5′-GGAAGCGCTTTCGGATG-3′ |
| Exon 9-11 | 1618 | 5′-CCCTGTGCTACGAGCTCAAC-3′ | 1678 | 5′-CACAGCTGGCTCCCTCAG-3′ |
| Exon 11-14 | 1638 | 5′-GCCATCCAGAACTCCATGA-3′ | 1640 | 5′-CAGGGATGTTCCAGTTCTGC-3′ |
| Exon 13-16 | 1677 | 5′-GAGTTCTACCGGGACTGGTG-3′ | 1641 | 5′-ATCATGCCGGTGAAGGC-3′ |
| Exon 16-17 | 1599 | 5′-CGAGTACCTGGTGAGCATCC-3′ | 1601 | 5′-TGTGCACAGCACTTTATTGAC-3′ |
| 5′end | 1755 | 5′-AGAAATGGGAAGTGCAGACC-3′ | 1738 | 5′-TGATGCCTACCTAAGCTCTACC-3′ |
| 5′end | 1754 | 5′-CAGGGTGGGATCACCTGAG-3′ | 1734 | 5′-TGGATTGCAAAGTCCTGTCC-3′ |
| 5′end | 1753 | 5′-GGTGGATGACGGGTAGAGG-3′ | 1716 | 5′-ACAGCTGGAGTGAGGACACC-3′ |
| 5′end | 1721 | 5′-TGAGGCCCTGATCTCTCAAC-3′ | 1709 | 5′-TGTCTTGGAGTAGCGTGTGG-3′ |
| 5′end | 1722 | 5′-AAGGGGATACTCCTGATCCAC-3′ | 1706 | 5′-AGGCCCCCACAGTAGACAAG-3′ |
| 5′end | 1723 | 5′-TCTGCAGATGAAGGCAGAAG-3′ | 1698 | 5′-CGCGCATACCTTTGTAGTCC-3′ |
| 3′end | 1711 | 5′-CCATGCTCTGGAAACCCTAC-3′ | 1718 | 5′-GCGGCAGAGCCAGTAGAG-3′ |
| 3′end | 1729 | 5′-TCAGCAGGTAGTTGGGTGTG-3′ | 1756 | 5′-CTCCCTGTCTGTTCCTCCTG-3′ |
| Intron 1 | 1866 | 5′-GACACCTGGTGCGTCCTTC-3′ | 1867 | 5′-GAGGGGAGCATTTCCCAATC-3′ |
| Intron 1 | 1868 | 5′-TACCCCCACAGACTGTCCTC-3′ | 1679 | 5′-GGCCACCATTCAAACCAC-3′ |
| Intron 2 | 1602 | 5′-GAATTGGTGTGTGGTGATGC-3′ | 1674 | 5′-CAGTCCTGCTCCCTCCAG-3′ |
| Intron 2 | 1673 | 5′-GCCACACTCTGCAGGACTC-3′ | 1671 | 5′-TGACAGGCTCAGAGATGCAG-3′ |
| Intron 2 | 1672 | 5′-TGGTAAGCTGGCTGGTTAGG-3′ | 1634 | 5′-TAGAAATAACCGTGCGTTGC-3′ |

TABLE 3
Results of PCR analysis of Genebridge 4 (GB4)
hamster-human radiation hybrid panel

| No. | Cell line | |
|---|---|---|
| | | PCR assay^{(a)} |
| 1 | 4A4 | 0 |
| 2 | 4A5 | 2 |
| 3 | 4AA5 | 1 |
| 4 | 4AA7 | 0 |
| 5 | 4B2 | 2 |
| 6 | 4B3 | 0 |
| 7 | 4B9 | 2 |
| 8 | 4BB1 | 0 |
| 9 | 4BB6 | 0 |
| 10 | 4BB8 | 1 |
| 11 | 4BB10 | 2 |
| 12 | 4BB12 | 2 |
| 13 | 4C3 | 1 |
| 14 | 4C11 | 0 |
| 15 | 4CC8 | 0 |
| 16 | 4D1 | 0 |
| 17 | 4D7 | 0 |
| 18 | 4DD2 | 0 |
| 19 | 4DD5 | 1 |
| 20 | 4DD8 | 0 |
| 21 | 4E2 | 1 |
| 22 | 4E4 | 0 |
| 23 | 4E6 | 0 |
| 24 | 4E11 | 0 |
| 25 | 4F6 | 1 |
| 26 | 4F7 | 1 |
| 27 | 4F13 | 0 |
| 28 | 4G1 | 0 |
| | | PCR assay |
| 29 | 4G5 | 0 |
| 30 | 4G6 | 0 |
| 31 | 4G7 | 0 |
| 32 | 4G11 | 1 |
| 33 | 4H1 | 0 |
| 34 | 4H8 | 0 |
| 35 | 4H9 | 1 |
| 36 | 4H12 | 0 |
| 37 | 4I1 | 0 |
| 38 | 4I4 | 1 |
| 39 | 4J2 | 0 |
| 40 | 4J5 | 0 |
| 41 | 4J9 | 0 |
| 42 | 4K5 | 0 |
| 43 | 4K7 | 1 |
| 44 | 4K8 | 2 |
| 45 | 4K9 | 0 |
| 46 | 4K12 | 1 |
| 47 | 4L3 | 1 |
| 48 | 4L4 | 0 |
| 49 | 4L6 | 0 |
| 50 | 4M4 | 0 |
| 51 | 4M5 | 1 |
| 52 | 4N3 | 0 |
| 53 | 4N5 | 0 |
| 54 | 4N6 | 0 |
| 55 | 4N7 | 0 |
| 56 | 4N12 | 1 |
| 57 | 4O5 | 0 |
| 58 | 4O10 | 2 |
| 59 | 4P2 | 0 |
| 60 | 4P9 | 0 |
| 61 | 4P11 | 0 |
| 62 | 4Q2 | 1 |
| 63 | 4Q4 | 0 |
| 64 | 4R1 | 0 |
| 65 | 4R2 | 0 |
| 66 | 4R3 | 0 |
| 67 | 4R5 | 0 |
| 68 | 4R6 | 0 |
| 69 | 4R10 | 1 |
| 70 | 4R12 | 2 |
| 71 | 4S3 | 1 |
| 72 | 4S6 | 0 |
| 73 | 4S10 | 2 |
| 74 | 4S12 | 0 |
| 75 | 4T3 | 0 |
| 76 | 4T4 | 0 |
| 77 | 4T10 | 0 |
| 78 | 4T11 | 0 |
| 79 | 4U1 | 1 |
| 80 | 4U3 | 2 |
| 81 | 4V2 | 2 |
| 82 | 4V3 | 1 |
| 83 | 4V7 | 0 |
| 84 | 4V8 | 0 |
| 85 | 4W1 | 0 |
| 86 | 4Y4 | 0 |
| 87 | 4Y8 | 0 |
| 88 | 4Y9 | 0 |
| 89 | 4Z5 | 0 |
| 90 | 4Z6 | 0 |
| 91 | 4Z9 | 1 |
| 92 | 4Z11 | 0 |
| 93 | 4Z12 | 0 |

^{(a)}0, negative; 1, positive; 2, not assayed

TABLE 4
Bovine ESTs identified in the EST database using the human DGAT mRNA
sequence (accession XM_005135) as input for BLASTN (Continued)

| | | | Position in bovine |
|---|---|---|---|
| Accession | Size (in bp) | Source of mRNA | DGAT^{(a)} |
| AW446908 | 479 | pooled tissue from lymph node, ovary, fat, | 256-780 (exon 2-9) |
| | | hypothalamus, and pituitary | |
| AW483961 | 205 | pooled tissue from day 20 and day 40 | 1594-1745 (3′UTR) |
| | | embryos | |
| AW486026 | 385 | pooled tissue from day 20 and day 40 | 1336-1720 (exon17-3′UTR) |
| | | embryos | |
| AW652329 | 542 | pooled tissue from lymph node, ovary, fat, | 990-1530 (exon 13-3′UTR) |
| | | hypothalamus, and pituitary | |
| BE664362 | 415 | pooled tissue from day 20 and day 40 | 1321-1735 (exon17-3′UTR) |
| | | embryos | |
| BE753833 | 422 | pooled tissue from testis, thymus, semiten- | 1369-1745 (exon17-3′UTR) |
| | | dono sus muscle, longissimus muscle, | |
| | | pancreas, adrenal, and endometrium | |
| BE664357 | 456 | pooled tissue from day 20 and day 40 | 1321-1745 (exon17-3′UTR) |
| | | embryos | |
| BE900091 | 527 | adipose tissue | 1097-1561 (exon14-3′UTR) |
| BE751071 | 475 | pooled tissue from testis, thymus, semiten- | 1087-1560 (exon14-3′UTR) |
| | | dono sus muscle, longissimus muscle, | |
| | | pancreas, adrenal, and endometrium | |
| AW446985 | 485 | pooled tissue from lymph node, ovary, fat, | 594-1143 (exon 7-11) |
| | | hypothalamus, and pituitary | |
| AW326076 | 141 | pooled tissue from lymph node, ovary, fat, | 703-772 (exon 8-9) |
| | | hypothalamus, and pituitary | |
| BE486748 | 174 | mammary tissues at eight physiological, | 906-986 (exon 11-12) |
| | | developmental, and disease states | |

^{(a)}Base 1 = first base of start codon

TABLE 5
Exon-intron structure of the bovine DGAT gene

| | | | | | | |
|---|---|---|---|---|---|---|
| | Position in | Size | | | Size | |
| Exon | bovine DGAT^{(a)} | (bp) | 5′-splice donor^{(b)} | Intron | (bp) | 3′-splice acceptor^{(b)} |

| | | | | | | |
|---|---|---|---|---|---|---|
| 1 | 1-191 | 191 | CCTGAGgtagcg | 1 | 3617 | ctccagGTGTCA |
| 2 | 192-279 | 88 | ATGCTGgtacgt | 2 | 1944 | tcgcagATCTTA |
| 3 | 280-320 | 41 | CATCAAgtgagt | 3 | 79 | ctgcagGTATGG |
| 4 | 321-406 | 86 | TCATTGgtgagc | 4 | 92 | cctcagTGGCCA |
| 5 | 407-459 | 53 | GCCGTGgtaagc | 5 | 215 | ccccagGGAGCT |
| 6 | 460-565 | 106 | CTCCAGgtgggc | 6 | 89 | ccacagTGGGCT |
| 7 | 566-679 | 114 | AGGCTGgtgagg | 7 | 100 | tcgtagCTTTGG |
| 8 | 680-754 | 75 | ACCGCGgtgagg | 8 | 70 | ttccagATCTCT |
| 9 | 755-858 | 104 | GAGATGgtgagg | 9 | 90 | ccccagCTGTTC |
| 10 | 859-897 | 39 | CAGCAGgtacgt | 10 | 60^{(c)} | ttgcagTGGATG |
| 11 | 898-939 | 42 | TTCAAGgtgagc | 11 | 73 | ccacagGACATG |
| 12 | 940-984 | 45 | CTGGCGgtgagt | 12 | 74 | ccacagGTCCCC |
| 13 | 985-1097 | 113 | CTGGTGgtgggt | 13 | 87 | ccgcagGAACTC |
| 14 | 1098-1163 | 66 | CATCAGgtgggt | 14 | 86 | ccgcagACACTT |
| 15 | 1164-1251 | 88 | CACGAGgtcagt | 15 | 81 | cctcagTACCTG |
| 16 | 1252-1314 | 63 | GCGCAGgtgagc | 16 | 72 | ccccagATCCCG |
| 17 | 1315-1470 | 156 | | | | |

^{(a)}Base 1 = first base of start codon
^{(b)}Exon sequences are indicated in upper case letters, intron sequences in lower case letters. The consensus splice site sequences are in boldface.
^{(c)}Intron 10 contains a (G)ₙ stretch that could not be resolved by sequencing.

TABLE 6
Panel of individual animals and animals belonging to a pool

| | Lab. no. | Herdbook no. | Breed | Sub-species^{(a)} |
|---|---|---|---|---|
| individual | FV19 | 7620 | Simmental | taurus |
| animals | FV27 | 25100 | Simmental | taurus |
| | FV28 | 50148 | Simmental | taurus |
| | SB26 | 790580 | Simmental | taurus |
| | SB37 | 102430 | Simmental | taurus |
| | SB45 | 252006 | Simmental | taurus |
| | AN1 | | Angus | taurus |
| | KE2 | | Kerry | taurus |
| | SA4 | | Sahiwal | indicus |
| | HA8 | | Hariana | indicus |
| SBpool | SB 2 | 102399 | Holstein-Friesian | taurus |
| | SB 9 | 790121 | Holstein-Friesian | taurus |
| | SB 13 | 790223 | Holstein-Friesian | taurus |
| | SB 14 | 790253 | Holstein-Friesian | taurus |
| | SB 22 | 790510 | Holstein-Friesian | taurus |
| | SB 33 | 790361 | Holstein-Friesian | taurus |
| | SB 41 | 790062 | Holstein-Friesian | taurus |
| | SB 43 | 790183 | Holstein-Friesian | taurus |
| | SB 44 | 102350 | Holstein-Friesian | taurus |
| | SB 47 | 102315 | Holstein-Friesian | taurus |

^{(a)}Bos taurus taurus or Bos taurus indicus

TABLE 7
Composition of DNA pools: Fleckvieh
(Bavarian Simmental) breed

| | | | | Breeding |
|---|---|---|---|---|
| Pool^{(a)} | Lab. no. | Herdbook no. | Name | value |
| FVpool32+ | 901 | 194100 | HASTROL | 0.83 |
| FVpool12+ | 902 | 195260 | PROLAP | 0.78 |
| | 903 | 50223 | LABTON | 0.77 |
| | 906 | 39910 | RAPID | 0.75 |
| | 907 | 169044 | HAGENT | 0.74 |
| | 910 | 178317 | LOCANDA | 0.71 |
| | 911 | 165011 | HAGER | 0.70 |
| | 912 | 7889 | ROLAND | 0.70 |
| | 1066 | 1146 | LOMBARD | 0.70 |
| | 913 | 34380 | ALPAN | 0.69 |
| | 914 | 187217 | HALLSTRAS | 0.69 |
| | 916 | 60535 | LAMBADA | 0.69 |
| | 917 | 60250 | PLANSEE | 0.69 |
| | 918 | 54474 | PROMO | 0.69 |
| | 919 | 172162 | LOMB | 0.68 |
| | 920 | 184506 | LOMO | 0.68 |
| | 921 | 169042 | HAGSON | 0.67 |
| | 922 | 172174 | LOMBOLO | 0.66 |
| | 923 | 178308 | LORETTO | 0.66 |
| | 924 | 165010 | HAGEL | 0.65 |
| | 925 | 22153 | RALBIT | 0.65 |
| | 926 | 645073 | ZEPTER | 0.65 |
| | 927 | 60527 | ALPIN | 0.64 |
| | 930 | 34554 | STREUSAND | 0.63 |
| | 932 | 187049 | HALLERTAU | 0.62 |
| | 933 | 21784 | UTNACH | 0.62 |
| | 935 | 187138 | HALBEM | 0.59 |
| | 936 | 175061 | HALLEM | 0.59 |
| | 937 | 191053 | HATARI | 0.59 |
| | 939 | 53535 | GAST | 0.58 |
| | 940 | 191045 | RODOS | 0.57 |
| | 942 | 50246 | FODA | 0.56 |
| FVpool32− | 1019 | 45432 | HONER | −0.31 |
| | 1021 | 53381 | PRO | −0.31 |
| | 1023 | 178075 | RAVELLI | −0.31 |
| | 1025 | 191283 | WALTL | −0.31 |
| | 1026 | 39733 | WESPE | −0.31 |
| | 1029 | 68130 | RAUDI | −0.33 |
| | 1032 | 27876 | HERMANUS | −0.34 |
| | 1033 | 21971 | HOPPE | −0.34 |
| | 1034 | 22043 | HOPURG | −0.34 |
| | 1035 | 60552 | HUMBACH | −0.34 |
| | 1036 | 68030 | ZAR | −0.34 |
| | 1038 | 22093 | PRONER | −0.35 |
| | 1039 | 184256 | RAUWOLF | −0.35 |
| | 1040 | 187114 | RIVA | −0.35 |
| | 1043 | 184280 | JUL | −0.36 |
| | 1046 | 53487 | BONWEIN | −0.37 |
| | 1047 | 53493 | PREUS | −0.37 |
| | 1048 | 68175 | RAMSES | −0.37 |
| | 1049 | 53607 | ROTWEIN | −0.37 |
| | 1050 | 53625 | PRODOMO | −0.38 |
| FVpool12− | 1051 | 176156 | RAFAEL | −0.38 |
| | 1053 | 27848 | WIND | −0.39 |
| | 1054 | 68040 | HIRTE | −0.41 |
| | 1055 | 53517 | WICHT | −0.41 |
| | 1056 | 7787 | WHISKY | −0.43 |
| | 1058 | 176009 | FREDL | −0.45 |
| | 1060 | 39860 | WIM | −0.46 |
| | 1061 | 53460 | WINZER | −0.46 |
| | 1062 | 53293 | ZECHER | −0.46 |
| | 1063 | 27847 | RENOIR | −0.47 |
| | 1064 | 68195 | RASTER | −0.51 |
| | 1065 | 27851 | WICKY | −0.51 |
| ^{(a)}The bulls were selected among 4070 artificial insemination bulls born 1990 and later. The mean breeding value fat % of the unselected bulls was 0.089, the standard deviation 0.217. Bulls with breeding values greater 0.5 (N = 154, mean = 0.646 ± 0.117) and smaller −0.3 (N = 89, mean = −0.380 ± 0.062) were selected. DNA samples could be obtained from 48 bulls on the positive side | | | | |
| # (mean = 0.647 ± 0.079) and 36 bulls on the negative side (mean = −0.381 ± 0.079). The mean breeding values (± standard deviations) of the pooled groups were as follows: FVpool12+, 0.729 ± 0.045; FVpool32+, 0.669 ± 0.063; FVpool32−, −0.381 ± 0.059; FVpool12−, −0.445 ± 0.042 | | | | |

TABLE 8
Composition of DNA pools: Braunvieh (Brown Swiss) breed

| Pool^{(a)} | Lab. no. | Herdbook no. | Name | Breeding value |
|---|---|---|---|---|
| BVpool20+ | 909 | 78780 | BREILORI | 0.73 |
| | 929 | 79030 | BREICON | 0.63 |
| | 943 | 340530 | EURO | 0.54 |
| | 951 | 79195 | VINCOL | 0.50 |
| | 952 | 79115 | EMOZ | 0.47 |
| | 953 | 348544 | STRIFMAN | 0.46 |
| | 954 | 78475 | DOTRAY | 0.45 |
| | 955 | 348105 | BRAY | 0.44 |
| | 956 | 349447 | BREIMORY | 0.42 |
| | 957 | 78635 | DOTION | 0.40 |
| | 959 | 77888 | ROMEIS | 0.38 |
| | 961 | 348247 | BREIZ | 0.37 |
| | 962 | 348591 | STRIZIN | 0.37 |
| | 964 | 349569 | HUCNOS | 0.35 |
| | 965 | 72695 | DOLEIN | 0.34 |
| | 966 | 340573 | BREISAD | 0.33 |
| | 967 | 340015 | STRELE | 0.32 |
| | 968 | 78980 | EMPIKT | 0.31 |
| | 971 | 79080 | RELVIN | 0.31 |
| | 972 | 78880 | BAYDOT | 0.29 |
| BVpool20− | 1004 | 78225 | DOBROY | −0.22 |
| | 1006 | 78200 | VISTAR | −0.22 |
| | 1007 | 348215 | CREVIN | −0.24 |
| | 1008 | 72625 | TRALAS | −0.24 |
| | 1009 | 348607 | VIVAT | −0.24 |
| | 1011 | 72680 | BAGAT | −0.27 |
| | 1012 | 72470 | SIRAY | −0.27 |
| | 1014 | 72930 | PETOS | −0.29 |
| | 1015 | 78090 | SIMPUR | −0.30 |
| | 1017 | 78470 | BARI | −0.31 |
| | 1018 | 78840 | BLESTRI | −0.31 |
| | 1024 | 78860 | RENZ | −0.31 |
| | 1027 | 78560 | JETSTRI | −0.30 |
| | 1028 | 72490 | JUP | −0.30 |
| | 1030 | 85550 | RESTOR | −0.30 |
| | 1037 | 78015 | DUKE | −0.40 |
| | 1042 | 78695 | CRAUTS | −0.40 |
| | 1044 | 348104 | PETMAN | −0.40 |
| | 1045 | 340010 | BAY | −0.40 |
| | 1057 | 78155 | JARGI | −0.40 |
| ^{(a)}The bulls were selected among 656 artificial insemination bulls born 1990 and later. The mean breeding value “fat %” of the unselected bulls was 0.006, the standard deviation 0.185. Bulls with breeding values greater 0.2 (N = 84, mean = 0.325 ± 0.108) and smaller −0.2 (N = 56, mean = −0.334 ± 0.101) were selected. DNA samples could be obtained from 54 bulls on the positive side | | | | |
| # (mean = 0.316 ± 0.111) and 22 bulls on the negative side (mean = −0.306 ± 0.055). The mean breeding values (±standard deviations) of the pooled groups were as follows: BVpool20+, 0.421 ± 0.113; BVpool20−, −0.305 ± 0.057. | | | | |

TABLE 9

Variable positions in and around DGAT and genotypes of individual
animals

| | | Animals | | | | | | | | | |
|---|---|---|---|---|---|---|---|---|---|---|---|
| Position | Variation | FV19 | FV27 | FV28 | SB26 | SB37 | SB45 | AN1 | KE2 | SA4 | HA8 |

| | | | | | | | | | | | |
|---|---|---|---|---|---|---|---|---|---|---|---|
| 1465-1554 | 4, 5, 6^{(a)} | 4, 4 | 4, 4 | 4, 4 | 5, 5 | 5, 6 | | 5, 6 | | 4, 4 | 5, 6 |
| 3343 | C-G | CC | GC | CC | CC | CC | CC | CC | | CC | CC |
| 3399 | T-G | TT | TT | TT | TT | TT | TT | TT | | GG | TG |
| 7232 | A-G | AA | AA | AA | GG | AA | AA | AA | | GG | GG |
| 8567 | A-G | | | | AA | | | | | | |
| 8607 | G-A | | | | GG | | | | | | |
| 9284 | T-C^{(b)} | | | | | | | | | | |
| 10147 | A-C | AA | AA | AA | AA | AA | AA | AA | | CC | AA |
| 10433 | G-A | GG | GG | GG | AA | AG | AG | GG | GG | AA | AA |
| 10434 | C-A | CC | CC | CC | AA | CA | CA | CC | CC | AA | AA |
| 10508-10512 | G5-G6 | G5G5 | G5G5 | G5G5 | G5G5 | G5G5 | G5G5 | G5G5 | G5G5 | G5G5 | G5G6 |
| ca. | PCR^{(c)} | − | − | − | + | + | + | + | +/− | + | + |
| 10800 | | | | | | | | | | | |
| 11030 | G-A | GG | GG | GG | AA | AG | AG | GG | GG | GG | AA |
| 11048 | C-T | TT | TT | TT | CC | CT | CT | TT | TT | CC | CC |
| 11993 | T-C | TT | TT | TT | CC | TC | TC | TT | TT | TT | TT |
| 12005 | A-C | AA | AA | AA | AA | AA | AA | AA | AA | CC | AA |
| 12036 | T-C | TT | TT | TT | TT | TT | TT | TT | TT | CC | TT |
| 12056 | A-G | AA | AA | AA | AA | AA | AA | AA | AA | GG | AA |
| 12136 | G-A | GG | GG | GG | GG | GG | GG | GG | GG | AA | GG |
| 13309 | G-Cb | | | | | | | | | | |

^{(a)}Number of repeats (AGGCCCCGCCCTCCCCGG)
^{(b)}Detected in pooled DNA (see Table 8)
^{(c)}Variable PCR amplification (+, PCR product; −, no or very weak PCR product)

TABLE 10

Repeat at position 1465-1554 and genotypes of
pooled samples

| | | |
|---|---|---|
| 4, 4^{(a)} | 4, 5^{(a)} | 5, 5^{(a)} |

| | | |
|---|---|---|
| FV12− | FV12+ | |
| FV32− | FV32+ | |
| | BV20− | BV20+ |

^{(a)}Number of repeats (AGGCCCCGCCCTCCCCGG)

TABLE 11
Allelic frequencies estimated from sequencing traces of pooled samples

| Position^{(a)} | Exchange | SBpool | FV12+ | FV12− | FV32+ | FV32− | BV20+ | BV20− |
|---|---|---|---|---|---|---|---|---|
| 3343 | C-G | 1 | 1 | 0.79 | 1 | 0.70 | 1 | 0.82 |
| 8567 | A-G | n.d. | n.d. | n.d. | 0.42 | 0 | n.d. | n.d. |
| 8607 | G-A | n.d. | n.d. | n.d. | 0 | 0.49 | n.d. | n.d. |
| 9284 | T-C | n.d. | n.d. | n.d. | 0.54b | 0.92d | 0.90b | 1b |
| 10433 | G-A | n.d. | 0.39^{(b)} | 1b | 0.46b | 1 | 0.90b | 1b |
| 10434 | C-A | n.d. | 0.36b | 1b | 0.41b | 1 | 0.93b | 1b |
| 11030 | G-A | n.d. | 0.68b | 1b | 0.64b | 1 | 1b | 1b |
| 11048 | C-T | n.d. | 0.48b | 0.20b | 0.48b | 0.26d | 0b | 0b |
| 11993 | T-C | 0.61 | 0.64 | 1 | 0.65 | 1 | 1 | 1 |
| 130309 | G-C | n.d. | n.d. | n.d. | 0.39 | 1 | 1 | n.d. |

^{(a)}Only positions with single base exchanges and that are variable within Bos taurus taurus
^{(b)}5% DMSO in PCR

TABLE 12
Genotypes of individual animals

| | | | Position (base) | | | |
|---|---|---|---|---|---|---|
| | | Breeding | 10433 | 10434 | | 11048 |
| Pool | Lab # | value | (A)^{(a)} | (A)^{(a)} | 11030 (A)^{(a)} | (C)^{(a)} |
| FV12p+ | 901 | 0.83 | 1 | 1 | 0 | 0 |
| | 902 | 0.78 | 0 | 0 | — | — |
| | 903 | 0.77 | 1 | 1 | 0 | 0 |
| | 906 | 0.75 | 2 | 2 | 2 | 2 |
| | 907 | 0.74 | 1 | 1 | 0 | 1 |
| | 910 | 0.71 | 1 | 1 | 0 | 0 |
| | 911 | 0.70 | 1 | 1 | 0 | 1 |
| | 912 | 0.70 | 1 | 1 | 1 | 1 |
| | 1066 | 0.70 | 1 | 1 | 0 | 2 |
| | 913 | 0.69 | 1 | 1 | 0 | 1 |
| | 914 | 0.69 | 0 | 0 | 0 | 0 |
| | 916 | 0.69 | 2 | 2 | 1 | 2 |
| Average/ | | | 0.5% | 0.5% | 0.18% | 0.45% |
| Frequency | | | | | | |
| FV32p+ | 917 | 0.69 | 2 | 2 | 0 | 1 |
| | 918 | 0.69 | 1 | 1 | 0 | 1 |
| | 919 | 0.68 | 1 | 1 | 0 | 1 |
| | 920 | 0.68 | 0 | 0 | 0 | 1 |
| | 921 | 0.67 | 0 | 0 | 0 | 0 |
| | 922 | 0.66 | 1 | 1 | 0 | — |
| | 923 | 0.66 | 0 | 0 | 0 | 1 |
| | 924 | 0.65 | 1 | 1 | 0 | 1 |
| | 925 | 0.65 | 1 | 1 | 1 | 1 |
| | 926 | 0.65 | 0 | 0 | 0 | 2 |
| | 927 | 0.64 | 1 | 1 | 1 | 1 |
| | 930 | 0.63 | 1 | 1 | 1 | 1 |
| | 932 | 0.62 | 2 | 2 | 0 | 1 |
| | 933 | 0.62 | 2 | 2 | 1 | 0 |
| | 935 | 0.59 | 1 | 1 | 0 | 1 |
| | 936 | 0.59 | 1 | 1 | 0 | 1 |
| | 937 | 0.59 | 1 | 1 | 0 | 1 |
| | 939 | 0.58 | 1 | 1 | 1 | 1 |
| | 940 | 0.57 | 0 | 0 | 0 | 1 |
| | 942 | 0.56 | 1 | 1 | 0 | 1 |
| Average/ | | | 0.47% | 0.47% | 0.15% | 0.94% |
| Frequency | | | | | | |

^{(a)}0, 1, 2, number of indicated allele; — assay failure

TABLE 13
F- and p-values of the variance analysis

| | |
|---|---|
| IMF_MLD | IMF_SEMI |

| Race | F-Vaule | Sig. | F-Value | P |
|---|---|---|---|---|
| Total | 0.19 | 0.827 n.s. | 3.47 | 0.040* |
| Holstein- | 0.36 | 0.704, n.s. | 5.35 | 0.013* |
| Friesian | | | | |
| Charolais | 0.15 | 0.703, n.s. | 1.13 | 0.301, n.s. |

TABLE 14
Least square means and standard error

| | | |
|---|---|---|
| | IMF_MLD | IMF_SEMI |
| | LSM +/− | LSM +/− |
| animals | s.e. | s.e. |

| | | | | |
|---|---|---|---|---|
| gesamt | | | | |
| L/L | 5.57 | 0.99 | 3.95 | 0.59 |
| L/A | 5.05 | 0.49 | 2.35 | 0.29 |
| A/A | 4.88 | 0.41 | 2.35 | 0.24 |
| Holstein-Friesian | | | | |
| L/L | 7.07 | 1.04 | 4.33 | 0.53 |
| L/A | 6.14 | 0.61 | 2.39 | 0.31 |
| A/A | 6.08 | 0.82 | 3.04 | 0.41 |
| Charolais | | | | |
| L/A | 3.80 | 0.62 | 2.46 | 0.50 |
| A/A | 3.53 | 0.32 | 1.85 | 0.26 |

### REFERENCES

Asil Memisoglu (2001) Pharmacogenomics, published on www.thebiotechclub.orq/Tech/pharmacogenomics.html.

Barendse W J (1999). Assessing lipid metabolism. International Publication Number WO 99/23248.

Coppieters W, Riquet J, Arranz JJ, Berzi P, Cambisano N, Grisart B, Karim L, Marcq F, Moreau L, Nezer C, Simon P, Vanmanshoven P, Wagenaar D, Georges M (1998) A QTL with major effect on milk yield and composition maps to bovine chromosome 14. Mammalian Genome 9: 540-4.

Ewing B, Green P (1998) Base-calling of automated sequencer traces using phred. II. Error probabilities. Genome Research 8: 186-194.

Ewing B, Hillier L, Wendl M C, Green P (1998) Base-calling of automated sequencer traces using phred. I. Accuracy assessment. Genome Research 8: 175-185.

Farese R V, Jr., Cases S, Smith S J (2000) Triglyceride synthesis: insights from the cloning of diacylglycerol acyltransferase. Curr Opin Lipidol 11: 229-34.

Georges M, Nielsen D, Mackinnon M, Mishra A, Okimoto R, Pasquino A T, Sargent L S, Sorensen A, Steele MR, Zaho X, Womack J E, Hoeschele I (1995) Mapping of quantitative trait loci controlling milk production in dairy cattle by exploiting progeny testing. Genetics 139: 907-920.

Goddard M E, Wiggans G R (1999). Genetic improvement of dairy cattle. The Genetics of Cattle. R. Fries and A. Ruvinsky. Wallingford, CABI Publishing: 511-37.

Gordon D, Abajian C, Green P (1998) Consed: a graphical tool for sequence finishing. Genome Research 8: 195-202.

Harlow and Lane (1988), “Antibodies, A Laboratory Manual”, CSH Press, Cold Spring Harbor.

Heyen D W, Weller J I, Ron M, Band M, Beever J E, Feldmesser E, Da Y, Wiggans G R, VanRanden P M, Lewin H A (1999) A genome scan for QTL influencing milk production and health traits in dairy cattle. Physiological Genomics 1: 165-175.

Higgins and Hames (eds.), “Nucleic acid hybridization, a practical approach”, IRL Press, Oxford 1985

Melani, C., Rivoltini, L., Parmiani, G., Calabretta, B., Colombo, MP. (1991) Inhibition of proliferation by c-myb antisense oligodeoxynucleotides in colon adenocarcinoma cell lines that express c-myb. Cancer Res. 51: 2897-2901

Nickerson D A, Tobe V O, Taylor S L (1997) PolyPhred: automating the detection and genotyping of single nucleotide substitutions using fluorescence-based resequencing. Nucleic Acids Res 25: 2745-51.

Rebeiz M, Lewin HA (2000) Compass of 47,787 cattle ESTs. Anim Biotechnol 11: 75-241.

Riquet J, Coppieters W, Cambisano N, Arranz JJ, Berzi P, Davis SK, Grisart B, Farnir F, Karim L, Mni M, Simon P, Taylor JF, Vanmanshoven P, Wagenaar D, Womack JE, Georges M (1999) Fine-mapping of quantitative trait loci by identity by descent in outbred populations: application to milk production in dairy cattle. Proc Natl Acad Sci USA 96: 9252-7.

Sambrook et al., (1989) “Molecular Cloning, A Laboratory Manual”; CSH Press, Cold Spring Harbor.

Scopes, (1982), Protein Purification, Springer-Verlag, N.Y.

Smith SJ, Cases S, Jensen DR, Chen HC, Sande E, Tow B, Sanan DA, Raber J, Eckel RH, Farese RV, Jr. (2000) Obesity resistance and multiple mechanisms of triglyceride synthesis in mice lacking Dgat. Nat Genet 25: 87-90.

Steinecke, (1995), Ribozymes, Methods in Cell Biology 50, Galbraith et al. eds Academic Press, Inc. 449-460.

Threadgill DW, Fries R, Faber LK, Vassart G, Gunawardana A, Stranzinger G, Womack JE (1990) The thyroglobulin gene is syntenic with the MYC and MOS proto-oncogenes and carbonic anhydrase II and maps to chromosome 14 in cattle. Cytogenetics and Cell Genetics 53: 32-36.

Velmala R J, Vilkki H J, Elo K T, de Koning D J, Maki-Tanila A V (1999) A search for quantitative trait loci for milk production traits on chromosome 6 in Finnish Ayrshire cattle. Anim Genet 30: 136-43.

Warren W, Smith T P, Rexroad C E, Fahrenkrug S C, Allison T, Shu C L, Catanese J, de Jong PJ (2000) Construction and characterization of a new bovine bacterial artificial chromosome library with 10 genome-equivalent coverage. Mamm Genome 11: 662-3.

Zhang Q, Boichard D, Hoeschele I, Ernst C, Eggen A, Murkve B, Pfister-Genskow M, Witte LA, Grignola F E, Uimari P, Thaller G, Bishop M D (1998) Mapping quantitative trait loci for milk production and health of dairy cattle in a large outbred pedigree. Genetics 149: 1959-73.

1. A nucleic acid molecule encoding a bovine acyl CoA:diacylglycerol transferase (DGAT) contributing to or indicative for low fat content of milk and to low meat marbling (intramuscular fat content) wherein said nucleic acid molecule is selected from the group consisting of:
(a) a nucleic acid molecule having or comprising the nucleic acid sequence of SEQ ID NO: 1;
(b) a nucleic acid molecule comprising the coding sequence of the polypeptide of SEQ ID NO: 2;
(c) a nucleic acid molecule the complementary strand of which hybridizes under stringent conditions to the nucleic acid molecule of (a) or (b), wherein said nucleic acid molecule has at the position corresponding to position 10433 and 10434 of the DGAT gene (SEQ ID NO: 1) a guanine and a cytosine residue; and
(d) a nucleic acid molecule the complementary strand of which hybridizes under stringent conditions to the nucleic acid molecule of (a) or (b), wherein said nucleic acid molecule has at the DGAT gene (SEQ ID NO: 1) position
(i) 3343 a cytosine, 10433 a guanine, 10434 a cytosine, 11030 a guanine, 11048 a cytosine and 11093 a thymine;
(ii) 3343 a cytosine, 10433 a guanine, 10434 a cytosine, 11030 a guanine, 11048 a thymine, and 11093 a thymine; or
(iii) 3343 a guanine, 10433 a guanine, 10434 a cytosine, 11030 a guanine, 11048 a thymine and 11093 a thymine.

2. The nucleic acid molecule of claim 1 which is mRNA, genomic DNA (gDNA) or cDNA which is derived from said mRNA by reverse transcription of said mRNA.

3. The nucleic acid of claim 2, wherein said gDNA is a gene.

4-43. (canceled)

44. A fragment of the nucleic acid molecule of claim 1 having at least 14 nucleotides wherein said fragment comprises nucleotide position 10433 and 10434 of SEQ ID NO: 1.

45. A nucleic acid molecule which is complementary to the nucleic acid of claim 1.

46. A vector comprising the nucleic acid molecule of claim 1.

47. The vector of claim 46 comprising regulatory elements for expression of said nucleic acid molecule.

48. A primer or primer pair, wherein the primer or primer pair hybridize under stringent conditions to the nucleic acid molecule of claim 1 comprising nucleotide position 10433 and 10434 of SEQ ID NO: 1 or the complement strand thereof.

49. A host cell which contains the expression vector of claim 47.

50. A method for production of a functional bovine DGAT or a functional fragment thereof comprising:
(a) culturing the host cell of claim 49 containing the expression vector, which comprises the nucleic acid molecule under conditions allowing the expression of the encoded polypeptide; and
(b) collecting the polypeptide from the culture.

51. A functional bovine DGAT polypeptide or a functional fragment thereof encoded by a nucleic acid molecule of claim 1.

52. An antibody which binds to an epitope of the polypeptide or fragment of SEQ ID NO: 2 the epitope comprising a alanine at position 232 but not to a polypeptide or a fragment of SEQ ID NO: 4 having a lysine at position 232.

53. An antibody which binds to an epitope of the polypeptide or fragment of SEQ ID NO: 4 the epitope comprising a lysine at position 232 but not to a polypeptide or a fragment of SEQ ID NO: 2 having a alanine at position 232.

54. A transgenic, non-human animal comprising at least the nucleic acid molecule of claim 1.

55. The transgenic, non-human animal of claim 54 wherein said animal belongs to cattle.

56. A method of testing a mammal for its predisposition for fat content of milk and/or its predisposition for meat marbling comprising analyzing the nucleic acid of a sample comprising the gene encoding a bovine acyl CoA:diacylglycerol transferase (DGAT) or a corresponding mRNA for nucleotide polymorphisms which are connected with said predisposition.

57. The method of claim 56, wherein the nucleic acid molecule analyzed is the gene encoding DGAT.

58. The method of claim 56 wherein said nucleic acid is DNA.

59. The method of claim 58 wherein said DNA is gDNA.

60. The method of claim 56 wherein said nucleic acid is cDNA which is derived from said mRNA by reverse transcription of said mRNA.

61. The method of claim 56, wherein the nucleotide polymorphisms are located in the coding region of the DGAT gene.

62. The method of claim 61 wherein the nucleotide polymorphisms in the coding region of the gene encoding DGAT result in substitution, deletion and/or addition of at least one amino acid in the amino acid sequence of the polypeptide which is encoded by said gene.

63. The method of claim 56 wherein said nucleic acid molecule has at the position corresponding to position 10433 and 10434 of the DGAT gene (SEQ ID NO: 1) a guanine and a cytosine residue which correlate with a predisposition for low fat content of milk and to low meat marbling.

64. The method of claim 63, wherein said nucleic acid molecule has at the position corresponding to position:
(a) 3343 a cytosine, 10433 a guanine, 10434 a cytosine, 11030 a guanine, 11048 a cytosine and 11093 a thymine;
(b) 3343 a cytosine, 10433 a guanine, 10434 a cytosine, 11030 a guanine, 11048 a thymine, and 11093 a thymine; or
(c) 3343 a guanine, 10433 a guanine, 10434 a cytosine, 11030 a guanine, 11048 a thymine and 11093 a thymine
which correlates with a predisposition for low fat content of milk and low meat marbling.

65. The method of claim 56 wherein said nucleic acid molecule has at the position corresponding to position 10433 and 10434 of the DGAT gene (SEQ ID NO: 3) two adenine residues which correlate with a predisposition for high fat content of milk and high meat marbling.

66. The method of claim 65, wherein said nucleic acid molecule has at the position corresponding to position:
(a) 3343 a cytosine, 10433 an adenosine, 10434 an adenosine, 11030 an adenosine, 11048 a cytosine and 11093 a thymine;
(b) 3343 a cytosine, 10433 an adenosine, 10434 an adenosine, 11030 an adenosine, 11048 a cytosine and 11093 a cytosine;
(c) 3343 a cytosine, 10433 an adenosine, 10434 an adenosine, 11030 a guanine, 11048 a cytosine and 11093 a thymine;
(d) 3343 a thymine, 10433 an adenosine, 10434 an adenosine, 11030 a guanine, 11048 a cytosine and 11093 a cytosine;
(e) 3343 a cytosine, 10433 an adenosine, 10434 an adenosine, 11030 a guanine, 11048 a cytosine, and 11093 a cytosine; or
(f) 3343 a thymine, 10433 an adenosine, 10434 an adenosine, 11030 a guanine, 11048 a cytosine, and 11093 a cytosine
which correlates with a predisposition for high fat content of milk and high meat marbling.

67. The method of claim 56 wherein the nucleotide polymorphisms are located in a region which is responsible for the regulation of the expression of the product of the gene encoding DGAT.

68. The method of claim 56 wherein the nucleotide polymorphisms are single nucleotide polymorphisms (SNP).

69. The method of claim 56, wherein said testing comprises hybridizing a nucleic acid complementary to the gene encoding DGAT as a probe under stringent conditions to the nucleic acid molecules comprised in said sample and detecting hybridization.

70. The method of claim 69 further comprising digesting the product of said hybridization with a restriction endonuclease and analyzing the product of said digestion.

71. The method of claim 69, wherein said probe is detectably labeled.

72. The method of claim 56, wherein said testing comprises determining the nucleic acid sequence of at least a portion of said nucleic acid.

73. The method of claim 72, wherein the determination of the nucleic acid sequence is effected by solid-phase minisequencing.

74. The method of claim 56 further comprising, prior to analyzing the nucleic acid, amplification of at least a portion of said nucleic acid.

75. The method of claim 74, wherein in the amplification reaction at least one of the primers employed in said amplification reaction is a primer that hybridizes under stringent conditions to the nucleic acid molecule comprising nucleotide position 10433 and 10434 of SEQ ID NO: 1 or the complement strand thereof, comprising assaying for an amplification product.

76. The method of claim 74 wherein said amplification is effected by or said amplification is the polymerase chain reaction (PCR).

77. The method of claim 56 wherein the nucleic acid is analyzed by the use of:
(a) a primer extension assay;
(b) a differential hybridization assay; and/or
(c) an assay which detects allele-specific enzyme cleavage.

78. A method of testing a mammal for its predisposition for fat content of milk and/or its predisposition for meat marbling comprising:
(a) preparation of a tissue sample from the subject;
(b) contacting the sample with the antibody of claim 12 or with a different antibody which binds to an epitope of the polypeptide or fragment of SEQ ID NO: 4 the epitope comprising a lysine at position 232 but not to a polypeptide or a fragment of SEQ ID NO: 2 having an alanine at position 232; and
(c) detecting whether a specific binding of said antibody to its antigen has occurred.

79. The method of claim 78 wherein binding of the antibody indicates a predisposition of the mammal for low fat content of milk and to low meat marbling.

80. The method of claim 78 wherein binding of the different antibody indicates a predisposition of the mammal for high fat content of milk and to high meat marbling.

81. The method of claim 56, wherein the sample is isolated from cloven hoofed animals.

82. The method of claim 81, wherein the cloven hoofed animals are cattle, buffalos, yaks or pigs.

83. A kit comprising at least the fragment of the nucleic acid molecule of claim 4 in one or more containers.
